(19) United States
(12) Patent Application Publication
(10) Pub. No.: US 2003/0134810 A1
(43) Pub. Date: Jul. 17, 2003

(54) Methods and compositions comprising biocompatible materials useful for the administration of therapeutic agents

(76) Inventors: Chris Springate, Vancouver (CA); John K. Jackson, Vancouver (CA); Charles Winternitz, Delta (CA); Helen M. Burt, Vancouver (CA)

Correspondence Address: GRAYBEAL, JACKSON, HALEY LLP, 155 - 108TH AVENUE NE, SUITE 350, BELLEVUE, WA, 98004-5901, US

(21) Appl. No.: 10/259,260

(22) Filed: Sep. 26, 2002

Related U.S. Application Data
(60) Provisional application No. 60/328,175, filed on Oct. 9, 2001.
(60) Provisional application No. 60/328,203, filed on Oct. 9, 2001.

(51) Int. Cl. A61K 48/00

(52) U.S. Cl. 514/44; 514/55

(57) ABSTRACT

Compositions and methods for in vivo delivery of pharmacologically active agents associated with polymeric biocompatible materials. Compositions comprising a first, negatively charged pharmacologically active agent such as an oligonucleotide and a polycationic polymer such as chitosan or chitosan derivatives, optionally in a pharmaceutically acceptable carrier the composition providing controlled release and/or protection from degradation of the first, negatively charged pharmacologically active agent when introduced into the body. The pharmaceutically acceptable carrier can be a polymer paste or gel which may contain a second pharmacologically active agent which may be an anti-inflammatory and/or an anti-proliferative agent. Methods of making and administering a controlled release and/or protective from degradation compositions for the delivery of a pharmacologically active agent, such as a nucleic acid, in combination with a polycationic polymer and in a pharmaceutically acceptable carrier, to a mammal in a pharmaceutically effective amount.

### CROSS-REFERENCE TO RELATED APPLICATIONS

The present application claims priority from U.S. provisional patent application No. 60/328,175, filed Oct. 9, 2001, and from U.S. provisional patent application No. 60/328,208, filed Oct. 9, 2001.

### BACKGROUND

One way to treat proliferative disorders, such as cancer, and inflammatory disorders, such as arthritis, is with oligonucleotide drugs (therapeutics) such as DNA or RNA used as antisense agents (ASOs), ribozymes, RNA inhibitors and immune modulating oligonucleotides. These oligonucleotide therapeutics can be specific and relatively non-toxic, and depending on the desired use, they can, generate lacking proteins or inhibit over-produced proteins.

The effective use of oligonucleotide therapeutics, however, is limited by ineffective delivery to the diseased tissues. Significant issues include oligonucleotide degradation, rapid removal, also known as clearance, of the oligonucleotide therapeutics from the disease site or organism, and the inability to get the product across the cell membranes of the target tissue, which inhibits the drug's work at sites inside cells. Degradation or catabolism and/or rapid removal or clearance of oligonucleotide therapeutics results in increased doses, increased duration of therapy, and increased cost to patients receiving these oligonucleotide therapeutics.

Another method of treating proliferative or inflammatory diseases is the use of cytotoxic anti-proliferative or anti-inflammatory drugs, such as well known, but toxic, cancer drugs such as methotrexate, cisplatin paclitaxel. Such drugs may be less specific than oligonucleotides and can have toxic side effects arising from overexposure of non-diseased tissues and underexposure of diseased cells due to the desire to minimize toxicities, which underexposure may allow the cells to up-regulate pro-survival proteins, which in turn increases the resistance of the cells to the cytotoxic drugs.

Thus, there has gone unmet a need for improved methods and compositions and the like for at least one of controlling the release of oligonucleotide therapeutics, decreasing the degradation or catabolism of oligonucleotide therapeutics and delivering oligonucleotide and anti-proliferative and/or anti-inflammatory drugs to desired sites in a manner that enhances disease treatment, such as by disease site targeting or reduced toxicities. The present systems and methods provide these and other advantages.

### SUMMARY

The compositions, systems, methods, etc., discussed herein provide controlled release and/or protective formulations such as a polycationic polymer such as chitosan complexed with negatively-charged therapeutics such as ASOs and other oligonucleotide therapeutics. The compositions can also include one or more additional pharmacologically active agents, such as an anti-proliferative or antiinflammatory drug. The compositions can also include one or more polymeric pastes or other carrier that comprise the polycationic polymer, negatively-charged therapeutic and optionally one or more additional pharmacologically active agents. Such compositions offer one or more of the following advantages: a) protect the therapeutic from degradative processes; b) maintain either locally or systemically effective concentrations of the therapeutic via controlled release, which avoids the classic peaks and troughs of plasma drug concentrations usually observed when rapidly-cleared drugs are repeatedly administered to the systemic circulation; c) decrease the administration frequency of oligonucleotide or other therapeutics; d) decrease the amount of oligonucleotide or other therapeutics administered to patients per dose and overall; e) decrease the toxicities or side effects due to oligonucleotide or other therapeutics in the body f) decrease the elimination of the therapeutics form the body; and, g) reduce the need for vectoring agents since the effective diffusion of the ASO therapeutics into the target cells can be achieved by the maintenance of product concentrations, for example by implanting the controlled release system close to the diseased tissues where a strong diffusion gradient can be achieved. If a second drug is included the controlled release of the system may also improve the efficacy or reduce the toxicity of the second drug.

In some embodiments the polycationic polymer and negatively charged therapeutic, optionally with an anti-proliferative or anti-inflammatory agent, and optionally with a polymeric carrier can be formulated as, or as a part of, an ointment, cream, lotion, gel, spray, foam, mousse, coating, wrap, paste, barrier, implant, microsphere, microparticle, film, or the like. Representative examples of polymeric carriers include poly(ethylene-co-vinyl acetate), polyurethane, polyanhydrides, polyorthoesters, copolymers of poly(lactic acid) and poly(ε-caprolactone), gelatin, polysaccharides such as, for example, chitosan and hyaluronic acid, collagen matrices, celluloses and albumen as well as derivatives, conjugates, copolymers and blends of these polymers. Representative examples of other suitable carriers include but are not limited to ethanol; mixtures of ethanol and glycols such as, for example, ethylene glycol or propylene glycol; mixtures of ethanol and isopropyl myristate or ethanol, isopropyl myristate and water; mixtures of ethanol and eineol or D-limonene (with or without water); glycols (for example, ethylene glycol or propylene glycol) and mixtures of glycols such as propylene glycol and water, phosphatidyl glycerol, dioleoylphosphatidyl glycerol, Transcutol®, or terpinolene; mixtures of isopropyl myristate and 1-hexyl-2-pyrrolidone, N-dodecyl-2-piperidinone or 1-hexyl-2-pyrrolidone.

In some embodiments the present invention provides a controlled release drug delivery compositions comprising at least one polycationic polymer complexed with at least one first negatively charged pharmacologically active agent to provide controllable release of at least the first negatively charged pharmacologically active agent when administered to a patient. The compositions can further comprise at least one pharmaceutically acceptable carrier or excipient and at least one pharmaceutically acceptable carrier or excipient that can further comprise at least a second pharmacologically active agent. (Unless expressly stated otherwise or clear from the context, all embodiments, aspects, features, etc., can be mixed and matched, combined and permuted in any desired manner.) The polycationic polymer can comprise chitosan and the first negatively charged pharmacologically active agent can comprise a negatively charged oligonucleotide, which can be at least one of an antisense oligonucleotide, ribozyme, oligonucleotide RNA inhibitor, immune modulating oligonucleotide and nonspecific oligonucleotide.

The chitosan-negatively charged oligonucleotide complex can be in the form of a solution, gel, sol, suspension, spray, mousse, lotion, cream, ointment, paste, slurry, particulate, microparticulate, microsphere, film or slab within The compositions. The chitosan-negatively charged oligonucleotide complex can be in the form of a particulate, microparticulate or microsphere within The compositions. The compositions can be a solution, gel, sol, suspension, spray, mousse, lotion, cream, ointment, paste, slurry, particulate, microparticulate, microsphere, film, slab, wrap, barrier or implant. The pharmaceutically acceptable carrier or excipient can be a polymeric carrier that provides controllable release of at least one of the second pharmacologically active agent and the first negatively charged pharmacologically active agent. The second pharmacologically active agent can comprise at least one of paclitaxel, docetaxol, mitoxantrone, cisplatin or methotrexate. The compositions can be sized and formulated for intraperitoneal, intraarticular, intraocular, intratumoral, perivascular, subcutaneous, intracranial, intramuscular, intravenous, periophthalmic, inside the eyelid, intraoral, intranasal, intrabladder, intravaginal, intraurethral, intrarectal, adventitial, oral, nasal, rectal, topical. The compositions can be sized and formulated to be injected through a syringe needle. The compositions can further comprise a cell permeation enhancing agent.

The compositions further provide protection of the first negatively charged pharmacologically active agent from degradation. The patient can be a mammal, human, cow, horse, sheep, dog or cat. The polycationic polymer-first negatively charged pharmacologically active agent complex can be an ionic complex. The polycationic polymer can comprise at least one of a polyaminoacid, polyquaternary compound, protamine, polyvinylpyridine, polythiodiethylaminomethyl-ethylene, poly-p-aminostyrene, polycationic carbohydrate, polyimine, polycationic polymer derivatized with DEAE, polycationic polymethacrylate, polycationic polyacrylate, polycationic polyoxethane, polyamidoamine, polylysine, polyhistidine and polycationic starch.

The first negatively charged pharmacologically active agent can be at least one of an anti-hepatitis agent, anti-diabetic, anti-ocular disease agent, anti-microbial, anti-viral, anti-fungal, anesthetic, anti-vascular disease agent, anti-restenotic, anti-stenotic, vasoconstrictor, vasodilator, cardiotonic, enzyme, anti-inflammatory, anti-post surgical adhesion agent, anti-psoriatic, anti-arthritic, anti-multiple sclerosis agent, anti-inflammatory bowel disease agent, hormone, bone metabolism controlling agent, hypotensive, hypertensive, sedative, anti-cancer agent, antihistamine, anti-tussive, vaccine, anti-neural disorder agent and asthma treatment.

The second pharmacologically active agent can be at least one of an anti-hepatitis agent, anti-diabetic, anti-ocular disease agent, anti-microbial, anti-viral, anti-fungal, anesthetic, anti-vascular disease agent, anti-restenotic, anti-stenotic, vasoconstrictor, vasodilator, cardiotonic, enzyme, anti-inflammatory, anti-post surgical adhesion agent, anti-psoriatic, anti-arthritic, anti-multiple sclerosis agent, anti-inflammatory bowel disease agent, hormone, bone metabolism controlling agent, hypotensive, hypertensive, sedative, anti-cancer agent, antihistamine, anti-tussive, vaccine, anti-neural disorder agent and asthma treatment.

Also provided are surgical devices suitable for implantation in a patient comprising the compositions. The surgical device can be a catheter, shunt, device for continuous subarachnoid infusion, feeding tube, solid implant to prevent surgical adhesion, uterine implant, artificial sphincter, periurethral implant, splint, ophthalmic implant, contact lens, plastic surgery implant, stent including an esophageal stent, gastrointestinal stent, vascular stent, biliary stent, colonic stent, pancreatic stent, ureteric stent, urethral stent, lacrimal stent, Eustachian tube stent, fallopian tube stent, nasal stent, sinus stents, tracheal stent or bronchial stent, or a port including a venous access device comprising an external tunneled catheter, implanted port, epidural catheter or central catheter (PICC).

Additionally provided are methods of manufacturing a controlled release drug delivery composition comprising complexing at least one polycationic polymer with at least one first negatively charged pharmacologically active agent to provide controllable release of at least the first negatively charged pharmacologically active agent when administered to a patient. The methods can further comprise mixing, blending, dissolving, associating or incorporating the polycationic polymer-first negatively charged pharmacologically active agent complex with at least one pharmaceutically acceptable carrier or excipient. The methods can also further comprise mixing, blending, dissolving, associating or incorporating the polycationic polymer-first negatively charged pharmacologically active agent complex with at least one pharmaceutically acceptable carrier or excipient that can further comprise at least a second pharmacologically active agent.

Also provided are methods of at least one of treating, preventing or inhibiting at least one of a proliferative disease or inflammatory disease comprising administering to a patient at least potentially having the disease a therapeutically effective amount of the compositions herein

These and other aspects, features and embodiments are set forth within this application, including the following Detailed Description and attached drawings. In addition, various references are set forth herein, including in the Cross-Reference To Related Applications, which discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIG. 1 is a graph of tumor volume in mice pursuant to tumor treatment using controls and a chitosan-ASO-second drug mix.

FIG. 2 is a graph of tumor volume in mice pursuant to tumor treatment using controls and a chitosan-ASO-second drug mix.

FIG. 3 is a graph of prostate specific antigen (PSA) plasma levels in mice pursuant to tumor treatment using controls and a chitosan-ASO-second drug mix.

FIG. 4 is a graph of tumor volume in mice pursuant to tumor treatment using controls and a chitosan-ASO-second drug mix.

### DETAILED DESCRIPTION

The present invention comprises pharmaceutically acceptable compositions and methods that effectively, controllably deliver negatively charged oligonucleotide drugs or other negatively charged medicaments. The compositions comprise a pharmacologically active agent, such as the oligonucleotide drug, and a polycationic polymer, such as chitosan, optionally in a controlled release polymeric carrier such as a polymeric paste. The compositions, etc., can also optionally controllably deliver a specific second drug, such as an anti-proliferative or anti-inflammatory drug, and can deliver still other desired treatment agents, such as, for example, peptides and proteins. The polycationic polymer, which optionally is a microparticulate component, binds or encapsulates the negatively charged therapeutic, which in turn provides a controlled release system, optionally a controlled release microparticulate compartment, for the oligonucleotide drug. The optional paste component may also contain an anti-proliferative or anti-inflammatory drug and represents the controlled release compartment for such drug(s). The various therapeutics may act individually or synergistically against the disease.

The compositions can be manufactured, for example, by encapsulating, binding, or otherwise complexing (e.g., via ionic interaction or binding), the negatively charged drug and the polycationic polymer, which can optionally be a microparticulate compartment. The optional anti-proliferative and/or anti-inflammatory drug is then dispersed or dissolved in the optional paste carrier. The polycationic polymeric and negatively charged therapeutic fraction is then optionally dispersed in or otherwise combined with the optional paste-anti-proliferative drug fraction to form a paste composition that can be either homogenous or heterogeneous. This paste can, provided that the drugs themselves are suitably stable, be stably stored in a syringe and represents a stable homogenous dispersion of both drugs. The formulation can be injected at room temperature or other desired temperature directly into (or proximal or close to) the diseased tissues, where the controlled release of the drugs can be effected over periods of hours to months or years depending on the required dose. It can be injected or otherwise administered subcutaneously, intramuscularly, intraperitoneally, intraarticularly, topically, intravenously, or otherwise as desired to other sites in the body, and can be administered from once a day, week, or month, or even every three months or otherwise as desired.

Definitions.

The following paragraphs provide definitions of some of the terms used herein. All terms used herein, including those specifically discussed below in this section, are used in accordance with their ordinary meanings unless the context or definition clearly indicates otherwise. Also unless indicated otherwise, except within the claims, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

“Anti-inflammatory agent/factor/drug” indicates any protein, peptide, chemical or other molecule that acts to inhibit inflammatory events. Examples of anti-inflammatory agents include topoisomerase inhibitors such as camptothecin, doxorubicin, etoposide, metadione and beta-laperchone, non-steroidal anti-inflammatory drugs (NSAIDS) such as diclofenac sodium (Voltaren®) and 5-aminosalicylic acid (Salofalk®).

“Anti-proliferative” agent/factor/drug indicates any protein, peptide, chemical or other molecule that acts to inhibit proliferative events. Examples of anti-proliferative agents include microtubule inhibitors such as vinblastine, vincristine, colchicine and paclitaxel, or other agents such as cisplatin.

“Antisense” or “antisense oligonucleotide” indicates strands of deoxyribonucleic acid (DNA) or ribonucleic acid (RNA) ranging from approximately 5 to 100 nucleotide bases in length which inhibit the translation of the messenger ribonucleic acid (mRNA) to protein. These agents may inhibit the up-regulation of genes in the body (that is, they may inhibit the production of proteins in the body). The antisense therapeutics may inhibit or prevent the production of specific proteins that are up-regulated or activated in the disease process. Antisense therapeutics may bind to a specific mRNA as part of their mechanism of action.

“Chitosan” indicates any compound or composition which is a derivative or analogue of chitin. This term also includes chitin and various derivatives of chitosan such as carboxymethylchitosan, oleoyl chitosan and pegylated chitosan (Carbomer, Inc., Westborough, Mass.). Chitosan is a linear polysaccharide composed of two monosaccharides linked by glycosidic bonds and is manufactured by deacylation of chitin. Chitosan is a mucoadhesive, biocompatible polymer that is commercially available in a range of molecular weights and degrees of deacylation. Because the molecule has a protonable primary amine on a side chain, chitosan has weak cationic properties (is positively charged). Because chitosan is only weakly positively charged at physiological pH (pKa=6.5) it may not be as toxic as highly charged cations such as cationic lipids or poly l-lysine. Chitosan is typically not soluble in water but may be dissolved in weak acids such as a 2% acetic acid solution, and the chitosan degrades in vivo under the action of enzymes such as lysozymes.

“Composition” as used herein should be understood to indicate a combination of multiple substances into an aggregate mixture.

“Controlled release” indicates the release of oligonucleotide therapeutics or other agents into the surrounding media or body in a selected time-dependent manner. The release can be from approximately several hours to several years.

“Drug,” “therapeutic agent,” “therapeutic,” and the like indicates any molecule that has a significant effect on the body to treat or prevent conditions or diseases.

“Gene” indicates strands of DNA which are expressed as one or more proteins in the body.

“Gene therapy agent/therapeutic/drug” indicates any oligonucleotide, gene, protein, peptide, chemical or other molecule that modulates the expression or function of a gene.

“Hydroxyapatite” (HAP) indicates a mineral of the chemical formula Ca₁₀(PO₄)₆OH₂ or similar analogue or derivative thereof.

“Immune modulating oligonucleotide” indicates strands of DNA or RNA ranging from approximately 5 to 100 nucleotide bases in length which act as a therapeutic agent in the body by modulating the immune system.

“Inflammatory disease/disorder” indicates any of the non-cancer, inflammatory diseases discussed herein.

“Medicament” indicates pharmaceutical compositions as well as any medical device, implant, or the like which is adapted to treat a disease. Therefore, an anti-proliferative or an anti-inflammatory medicament includes pharmaceutical compositions that treats the disease, as well as medical devices, implants, and the like adapted, for example by incorporation of an anti-proliferative agent and an oligonucleotide therapeutic, for treatment of such disease.

“Oligonucleotide” indicates strands of DNA or RNA or mixtures thereof from approximately 5 to 100 nucleotide bases.

“Oligonucleotide therapeutic/agent/drug” includes ASOs ribozymes, oligonucleotide RNA inhibitors, as well as immune modulating oligonucleotides. Oligonucleotide agents may, for example, be manufactured synthetically in the laboratory using well-known methods. Molecules other than nucleotide bases containing potential hydrogen bond sites can be used in place of nucleotides.

“Pharmacologically active agent” means any of a drug, therapeutic, agent, pro-drug or diagnostic.

“Polymer” indicates any molecule made up of a number of repeating units. Representative examples of polymers include poly(ethylene-co-vinyl acetate), poly(lactic acid), poly(glycolic acid), poly(ε-caprolactone), poly(ethylene glycol), pluronics, polyvalerolactone, polyanhydrides, polysaccharides, polyorthoesters, and copolymers, derivatives and blends thereof. Polymers can have a molecular weight ranging from about 100 Daltons to greater than about 500,000 Daltons. Polymers can be formed into films between about 10 μm and 2 mm thick. Polymers can be prepared in a variety of “paste” or gel forms and can be thermologically active, such that the polymers have different properties at different temperatures. For example, the polymers can be liquid at one temperature (for example, above about 37° C. or 40° C.) and solid or semi-solid at or below another temperature (for example, at ambient temperature or below about 37° C.), or liquid or semi-liquid at room temperature but set to a semi-solid or solid (for example for use as an implant) in aqueous media at another temperature (for example, 37° C.).

“Polymeric drug delivery” indicates the incorporation of oligonucleotide, anti-proliferative, and/or anti-inflammatory agents in a polymer or mixture of polymers so that the agents remain in a non-degraded form in the polymer and optionally are released from the polymer in a controlled manner over a period of time. Such polymeric formulations are known and can be manufactured from biodegradable, non-biodegradable, or water-soluble polymers and can be fashioned in a variety of forms including, for example, rod-shaped devices, pellets, slabs, capsules, films, pastes, gels, microspheres, sprays, foams or coatings on implantable medical devices.

“Proliferative disease/disorder” indicates any of the cancer and other proliferative diseases discussed herein.

“Ribozyme” indicates strands of DNA or RNA ranging from approximately 5 to 50 nucleotide bases in length that cleave mRNA and thereby inhibit the translation of the mRNA acid to protein. These agents may inhibit the up-regulation of genes in the body (that is, they may inhibit the production of proteins in the body).

The scope of the present systems and methods, etc., includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

Other terms and phrases in this application are defined in accordance with the above definitions, and in other portions of this application.

General Discussion of Certain Embodiments

Turning to some aspects of the invention, the present systems, etc., provide a polycationic polymer, optionally as a microparticulate, controlled release drug delivery composition or system for the delivery of negatively therapeutics to diseased tissues. The release of intact negatively charged therapeutics can be controlled by the charge interaction between the polycationic polymer and the therapeutic. The system can be used for the controlled delivery of negatively charged hydrophilic drugs such as negatively charged oligonucleotides and other active agents with negative charges such as negatively charged peptides and proteins. Chitosan will typically be used as the example of a polycationic polymer in the discussions below but the systems can also be used with other appropriate polycationic polymers. ASOs will typically be used as the example of a negatively charged therapeutic in the discussions below but the systems can also be used with other appropriate negatively charged therapeutics. Optionally, the polycationic polymer and negatively charged therapeutic may be in the form of a particulate or microparticulate.

One system comprises a polymeric injection vehicle containing or otherwise complexed with the chitosan-ASO, which composition can be injected into, or adjacent to, diseased tissues. The chitosan-ASO product can be either homogenous or heterogeneous. The polymer vehicle is biodegradable and biocompatible and allows for the chitosan-ASO to be located (and held) at the target site while reducing their removal from the site by enzymatic degradation, lymphatic drainage or phagocytic removal. The rate of release of the ASO from the chitosan can be controlled (and customized to fit a prescribed dosing regimen) by adjusting the ratio of ASO to chitosan. For example, at lower ratios of ASO to chitosan (where many chitosan binding sites may exert strong binding of ASO) the release rate is slow. On the other hand at high ratios of ASO to chitosan (where binding may be weaker) the system can provide a fast “quick release” or “burst phase” release of loosely bound ASO, which can also be followed by a steady release of moderately bound ASO. “Quick release” compositions release greater than approximately 10% w/w of at least one of the oligonucleotide therapeutic, and optionally, anti-proliferative agent and/or anti-inflammatory agent over a period of approximately five to fifteen days. Such “quick release” compositions can, in certain embodiments, release chemotherapeutic levels of the desired agent(s). In other embodiments, “slow release” compositions release less than approximately 10% w/w of the agent(s) from about five to fifteen days. The compositions can be stable for several months in storage and can be produced and/or maintained under sterile conditions.

The compositions discussed herein can be prepared for a variety of applications. For example, for administration to the cornea, the polymeric carrier may comprise muco-adhesive polymers such as poly(acrylic acid) polymers such as Carbopol®, dextran, hyaluronic acid, polymethacrylates or starch. See LeYung and Robinson, J. Controlled Release 5:223 (1988).

In one aspect, the present invention provides controlled release drug delivery compositions comprising at least one polycationic polymer, which can be a microparticulate, complexed with at least one first, pharmacologically active agent, which can be anionic, to provide at least one controlled release polycationic polymer compartment, which can be a microparticulate compartment, that controllably releases the first pharmacologically active agent when administered to a patient, the controlled release microparticulate compartment complexed with at least one controlled release polymeric carrier that further controllably modulates the release of the first pharmacologically active agent from the composition. The microparticulate polycationic polymer can comprise chitosan, the first pharmacologically active agent can comprise an oligonucleotide therapeutic and the composition can further comprise at least one second pharmacologically active agent comprising at least one of an anti-proliferative drug and an anti-inflammatory drug, wherein the composition controllably modulates the release of the second pharmacologically active agent from the composition.

The oligonucleotide therapeutic can comprise an antisense oligonucleotide, ribozyme, immune modulating oligonucleotide, or other oligonucleotide as desired. The microparticulate polycationic polymer can encapsulate, bind, ionically complex, covalently complex, or otherwise complex to the first anionic pharmacologically active agent.

The second pharmacologically active agent can comprise at least one of paclitaxel, methotrexate, and can controllably release chemotherapeutic levels of the second pharmacologically active agent. The second pharmacologically active agent can also comprise at least one of an anti-diabetic, antimicrobial, anesthetic, vasoconstrictor, vasodilator, cardiotonic, enzyme, anti-inflammatory, hormone, bone metabolism controlling agent, hypotensive, sedative, anti-cancer agent, antihistamine, antitussive, vaccine, and asthma treatment.

The composition, the microparticulate or the controlled release polymeric carrier can be a paste, homogenous or non-homogenous, ointment, cream, capsule, lotion, gel, spray, foam, mousse, coating, wrap, barrier, implant, microsphere, or film, which film can be less than about 2 mm thick comprising a tensile strength greater than about 70 N/cm². The paste or other form can encapsulate the controlled release microparticulate compartment. The microparticulate polycationic polymer can comprise porous microparticles, and the controlled release microparticulate compartment can be micronized.

The composition can be formulated to release greater or less then about 10% w/w of the oligonucleotide therapeutic and the second pharmacologically active agent over a period of about five to fifteen days. The composition can be sized and formulated for oral, nasal, rectal, intravenous, intraperitoneal, intramuscular, subcutaneous, or intraarticular, topical administration to a patient, and can be administered intra-tumorally into a tumor. The composition can be injected through a syringe needle, sprinkled on an open wound or surgical site, or otherwise applied as desired. The composition can also be administered by implanting a surgical device comprising the composition into a desired location.

In some embodiments, the composition can comprise, or can exclude, a cell permeation enhancing agent. The composition can further comprise at least one phosphate ion source able to provide a mildly alkaline local environment relative to an in vivo environment. The microparticulate polycationic polymer can comprise at least one of a polyaminoacid, polyquaternary compound, protamine, polyvinylpyridine, polythiodiethylaminomethyl-ethylene, poly-p-aminostyrene, polycationic carbohydrate, polyimine, polymer derivatized with DEAE, polymethacrylate, polyacrylate, polyoxethane, polyamidoamine, polylysine, polyhistidine and cationic starch.

In another aspect, the present invention provides pharmaceutical compositions comprising a pharmaceutically effective amount of chitosan ionically complexed with a pharmaceutically effective amount of at least one oligonucleotide therapeutic having less than about 100 nucleotides, the composition further comprising at least one of a pharmaceutically acceptable adjuvant, excipient, buffer and diluent, wherein the composition can be formulated to controllably modulate the release of the oligonucleotide from the composition. Such compositions can further comprise at least one pharmaceutically acceptable controlled release polymeric carrier that further modulates the release of the first pharmacologically active agent, and if desired a second pharmacologically active agent comprising at least one of an anti-proliferative drug and an anti-inflammatory drug, and wherein the composition controllably modulates the release of the second pharmacologically active agent from the composition.

In an further aspect, the present invention provides a controlled release drug delivery composition comprising at least one microparticulate polycationic polymer complexed with at least one first, anionic pharmacologically active agent to provide at least one controlled release microparticulate compartment that controllably releases the first pharmacologically active agent when administered to a patient, the controlled release microparticulate compartment complexed with at least one controlled release polymeric carrier complexed with at least one second pharmacologically active agent, the controlled release polymeric carrier modulating the release of the first and second pharmacologically active agents from the composition, and wherein the composition can further comprise at least one phosphate ion source able to provide a mildly alkaline local environment relative to an in vivo environment.

The microparticulate polycationic polymer can comprise chitosan, the first pharmacologically active agent can comprise an oligonucleotide therapeutic and the second pharmacologically active agent can comprise at least one of an anti-proliferative drug and an anti-inflammatory drug, wherein the composition controllably modulates the release of the second pharmacologically active agent from the composition.

In an further aspect, the present invention provides surgical devices suitable for implantation in a patient, the surgical devices comprising, for example coated in or made of, a controlled release drug delivery composition as discussed herein. The surgical device can be a stent, catheter, port, shunt, device for continuous subarachnoid infusion, feeding tube, solid implant to prevent surgical adhesion, uterine implant, artificial sphincter, periurethral implant, splint, ophthalmic implant, contact lens, plastic surgery implant or other device as desired. A suitable stent can be an esophageal stent, gastrointestinal stent, vascular stent, biliary stent, colonic stent, pancreatic stent, ureteric stent, urethral stent, lacrimal stent, Eustachian tube stent, fallopian tube stent, nasal stent, sinus stents, tracheal stent, or bronchial stent. The surgical device can also be a venous access device comprising an external tunneled catheter, implanted port, epidural catheter or central catheter (PICC).

In still another further aspect, the present invention provides kits comprising a composition as discussed herein in a pharmaceutically acceptable container, such as a syringe or a vial. The kits can comprise a surgical device as discussed herein in a pharmaceutically acceptable container. The kits can further comprise a notice associated with the container, the notice typically in a form prescribed by a governing agency regulating the composition, and can further comprise instructions about at least one of use of the composition, dosing a patient and mode of administration.

In still yet another further aspect, the present invention provides methods of manufacturing a controlled release drug delivery composition comprising: a) complexing at least one microparticulate polycationic polymer with at least one first, anionic pharmacologically active agent to provide at least one controlled release microparticulate compartment that controllably releases the first pharmacologically active agent when administered to a patient; b) complexing the controlled release microparticulate compartment with at least one controlled release polymeric carrier that further controllably modulates the release of the first pharmacologically active agent from the composition.

The microparticulate polycationic polymer can comprise chitosan, the first pharmacologically active agent can comprise an oligonucleotide therapeutic and the methods can further comprise complexing the composition with at least one second pharmacologically active agent comprising at least one of an anti-proliferative drug and an anti-inflammatory drug such that the composition controllably modulates the release of the second pharmacologically active agent from the composition. The methods can further comprise adding the composition to a surgical device suitable for implantation in a patient.

In a further aspect, the present invention comprise methods of making a pharmaceutical composition comprising ionically complexing a pharmaceutically effective amount of chitosan with a pharmaceutically effective amount of at least one oligonucleotide therapeutic having less than about 100 nucleotides, the composition further comprising at least one of a pharmaceutically acceptable adjuvant, excipient, buffer and diluent, wherein the composition can be formulated to controllably modulate the release of the oligonucleotide from the composition. The composition can further comprise at least one pharmaceutically acceptable controlled release polymeric carrier that further modulates the release of the first pharmacologically active agent, and the compositions can further comprise at least a second pharmacologically active agent comprising at least one of an anti-proliferative drug and an anti-inflammatory drug, and wherein the composition controllably modulates the release of the second pharmacologically active agent from the composition.

In another aspect, the present invention provides methods of controlling release of a pharmacologically active agent from a controlled release pharmaceutical composition comprising a pharmaceutically effective amount of chitosan ionically complexed with a pharmaceutically effective amount of at least one oligonucleotide therapeutic having less than about 100 nucleotides, the composition further comprising at least one of a pharmaceutically acceptable adjuvant, excipient, buffer and diluent, the method comprising adjusting the ratio of chitosan to oligonucleotide therapeutic to provide a desired rate of release. The composition can further comprise a pharmaceutically acceptable controlled release polymeric carrier that further modulates the release of the first pharmacologically active agent, and a second pharmacologically active agent comprising at least one of an anti-proliferative drug and an anti-inflammatory drug, and the composition can controllably modulate the release of the second pharmacologically active agent from the composition.

In still yet another further aspect, the present invention provides isolated and purified compositions as discussed herein for use in the manufacture of a medicament for inhibiting, preventing or treating a proliferative or inflammatory disease in a human patient. Also provided are methods of manufacturing a medicament able to reduce symptoms associated with proliferative or inflammatory disease in a human patient, comprising combining a pharmaceutically effective amount of a composition as discussed herein, and a pharmaceutically acceptable adjuvant, excipient, buffer or diluent. The disease can be, for example, cancer, arthritis, psoriasis, and surgical adhesion.

Polycationic Polymer-Pharmacologically Active Agent Mixture

The compositions include a complex of a polycationic polymer, optionally as a microparticulate component, and a negatively, charged pharmacologically active agent, which can be an ionic complex, covalent complex or other complex as desired, and can optionally be isolated in solid form. The microparticulate component has areas of positive charge which provides for the binding or complexation of negatively charged gene therapy agents or other desired, negatively charged agents. The gene therapy agents or other drugs can be volume enclosed within polymeric microspheres or microparticles manufactured from biocompatible polymers including those discussed herein. The gene therapy agents or other drugs can be volume enclosed within the microparticle so that the control of release is governed by the rate of erosion or degradation of the microparticle, or can be volume enclosed within porous microparticles so that the control of release is governed by the rate of diffusion of the gene therapy agents from within the porous microparticle.

The polycationic polymer can comprise one or more of chitosan, chitosan salt, chitosan derivative, chitin, polyaminoacids, polyquaternary compounds, protamine, polyvinylpyridine, polythiodiethylaminomethyl-ethylene, poly-p-aminostyrene, polycationic carbohydrates, polyimines, polymers derivatized with DEAE, polymethacrylates, polyacrylates, polyoxethanes, polyamidoamines, polylysine, polyhistidine, cationic starches, and derivatives or copolymers thereof. As noted above, chitosan and ASO will typically be discussed herein but the discussion includes other polycationic polymers as well.

In some embodiments the microparticulate fraction contains certain nucleotide bases that may bind or complex oligonucleotide or other types of gene therapy agents. The levels of binding and release can be controlled by customizing the sequence of nucleotide bases within the microparticulate-binding fraction. Other agents can also included in the composition that disrupt the binding interaction between the complementary bases. These disrupting agents are released within the composition in a controlled manner to allow for a secondary controlled release of the gene therapy agent.

Exemplary methods of making the chitosan-ASO include mixing approximately two-thirds of a part by weight sodium chloride with approximately two parts by weight chitosan. This mixture can be milled to reduce the particle size of the mixture to approximately 1-30 μm in diameter. The chitosan and sodium chloride mixture is then placed in a vial. In a separate vial one part by weight (compared to the chitosan and sodium chloride weights) ASO is dissolved in water to make an approximately 5-15% w/w solution. The ASO solution is then mixed with the chitosan and sodium chloride mixture and the chitosan allowed to swell or dissolve. The contents are then allowed to dry overnight at 37° C.

The composition can also be prepared by soaking chitosan particles with a concentrated solution of ASO then rapid drying so that binding of the DNA or RNA to the chitosan occurs. Such particles controllably release the ASO over a period of days to weeks. In some embodiments, the chitosan forms a microparticulate compartment and with the ASO forms a microparticulate:gene therapy fraction.

In some applications, a swollen (aqueous) chitosan gel or suspension containing ASO in solution can be injected into a body compartment. This gel can be injected directly into a disease site such as, for example, a tumor (cancer), or a synovial joint (arthritis), or around a blood vessel (restenosis). The gel/suspension can also be injected into any body compartment to act as a slow release depot of the ASO for systemic release of the ASO. Alternatively, the gel/suspension can be injected directly into the blood stream for release of the ASO into the systemic circulation. The particle size of the chitosan-ASO determines the therapeutic application of the intravenous administration. Very small (less than 10 μm) particles can be used for continuous circulation applications. Larger particles (or microspheres containing smaller chitosan-ASO particles) can be injected into an artery leading to a disease site (e.g., hepatic artery to a hepatic tumor), so that the particles embolize the blood flow in the capillary network of the diseased tissue. Such an embolization may serve two purposes: (1) it may cut off the supply of nutrients to the diseased tissue and inhibit the proliferative aspect of the disease or (2) the embolic material may release a therapeutic agent (such as an ASO) in a controlled manner at the disease site (termed chemoembolization).

The aqueous gel/suspension of chitosan-ASO may contain a viscosity enhancing agent, such as hyaluronic acid, gelatin or alginate/calcium, for example to slow down the dispersion or phagocytic removal of the chitosan-ASO particles from the diseased site and to slow the rate of release of the ASO. For example, the chitosan-ASO particles can be suspended at any concentration in a 2% hyaluronic acid gel (cross-linked with carbodiimide) and injected into the peritoneal cavity or other suitable location for the treatment of tumor resection sites (to prevent tumor regrowth). In this example, the mucoadhesive hyaluronic acid would adhere as a thin film to the resection site and hold the chitosan-ASO particles in the area for two days. The mucoadhesive properties of chitosan may also facilitate binding to membranes in vivo.

The weight of the polycationic polymer can be about 0.5, 1, 2, to 4 times the weight of the negatively charged pharmacologically active agent.

Generally, the ASOs released from the chitosan (or chitosan-polymer composite) can transfer into target cells without permeation enhancing agents, or per, for example via high local sustained concentrations of the antisense molecules to provide a diffusion gradient transfer into the cells. Akthar, et al., Trends in Cell Biology, 2: 139-144 (1992); Fell. P. L., et al., Antisense Nucleic Acid Drug Development, 7: 319-326 (1997). The methods, compositions, etc., herein provide adequate concentrations of desired substances for diffusion gradients to be effective. The chitosan can be initially micronized (particle size reduced to sub-micron size) before the oligonucleotide therapeutics are bound to the surface of the chitosan. This enhances entry of the chitosan-ASO particle to the inside of the cell.

If desired, however, permeation enhancers can be included in the locally applied formulation or otherwise as desired. Suitable permeation enhancers include diblock and triblock copolymers, detergents, positively charged molecules (such as, poly-l-lysine, etc.), p-glycoprotein inhibitors (such as pluronic copolymers, cyclosporin and verapamil), membrane fluidity modulating agents (such as amphipathic or membrane permeable molecules), and agents that carry the drugs across the cell membranes (such as cationic lipids or polymers). These permeation-enhancing agents can be a) directly bound to the drugs or microparticulate, b) dissolved or suspended in the polymeric carrier, c) bound, complexed or encapsulated in a secondary microparticulate fraction, or otherwise combined with the drugs or chitosan, to allow for the controlled release of these permeation-enhancing agents.

The microparticulate fraction such as chitosan, containing the complexed gene therapeutic can have a dimension (size) that is amenable to pinocytosis or endocytosis by cells so that the microparticulate is taken up by the diseased cells via such mechanisms. The microparticulate fraction can also comprise a substance that is less repelled by the surface charge of the cell membrane so that effective binding of the microparticulate to the cell is less inhibited by surface charge repulsion. The anti-proliferative, anti-inflammatory or other drugs, (discussed elsewhere herein) may accumulate in the membranes of the target cells causing a permeabilization effect that promotes the diffusion of the gene therapy agent, or other first drug, across the membrane. Such an accumulation of the anti-proliferative drug can be enhanced by the use of a drug efflux transporter (e.g., p-glycoprotein) inhibitors (e.g., pluronics, cyclosporin or verapamil) in the composition.

The negatively charged pharmacologically active agent and polycationic polymer can be in the form of particulates, microparticulates, microspheres, powders, dispersions, gels, solutions, suspensions, slurries, pastes, or other forms as desired.

Polymer Carrier.

The polycationic polymer-negatively charged therapeutic complexes, optionally as microparticulates or other forms as listed in the paragraph above, can be combined with (e.g., coated with or encapsulated within), a secondary matrix or polymer carrier to slow down the rate of release of the negatively charged therapeutic from the composition. The matrix can be a polymeric carrier, which can be a single polymer or blend of polymers, and when a paste or gel can form a semi-solid or waxy solid when introduced into an aqueous media or the body. The polymeric matrix can also, or alternatively, contain a small molecule drug or other desired active agent (for example, an anti-proliferative or an anti-inflammatory agent), that also has therapeutic efficacy against the disease or has some other desired effect on the target. The therapeutic agents may act individually or synergistically against the disease or other targets. The polycationic polymer-negatively charged therapeutic complex, for example, as chitosan-ASO, can be physically blended into the polymeric paste at approximately 40° C. at approximately 0.1-50% w/w chitosan and ASO particles.

In some embodiments, the polycationic polymer-negatively charged therapeutic complex, for example as chitosan-ASO, with or without a permeation-enhancing agent can be “hidden” from the immune system by the polymeric carrier. This can decrease the inflammatory responses by the body to the microparticulate component, increase the amount of negatively charged therapeutic that interact with the diseased cells, decrease the frequency of administration of the negatively charged therapeutic and anti-proliferative or anti-inflammatory agent, decrease the amount of gene therapy agent and anti-proliferative or anti-inflammatory agent administered to the patient, and decrease the side effects or toxicities of these agents to the patient.

This secondary matrix may take the form of, for example, biodegradable, biocompatible, polymeric coatings, microspheres or films. As discussed further elsewhere, a secondary phosphate ion (or other anion or cation) source can be incorporated into such a matrix to further control the release rate of ASOs from the chitosan. The ion source (e.g., sodium phosphate dibasic) creates a mildly alkaline environment since acid environments may be degradative to oligonucleotide products, and the positive charge on the amine group of chitosan (which binds the ASO) may be reduced by modulating the pH, which controls the release rate.

A polymeric paste can be prepared by physically blending at approximately 40° C. a waxy polymer such as poly(L-lactide) 2000 MW such as solid/waxy biodegradable triblock polymer of poly(DL-lactide-co-caprolactone) (PLC) and poly(ethylene glycol) (PEG) (with a final triblock copolymer structure of PLC-PEG-PLC, abbreviated as TB) with a liquid polymer such as methoxy-poly(ethylene glycol) 350 MW. Approximately 60% w/w waxy polymer and approximately 40% wlw liquid polymer make the paste injectable. An additional therapeutic such as an antiproliferative or anti-inflammatory drug such as paclitaxel can be dispersed, dissolved or suspended at a preferred concentration in the polymeric paste prior to the mixing with the chitosan-ASO therapeutic particles. The anti-proliferative or anti-inflammatory agent and the polymeric carrier can form a polymeric carrier:anti-proliferative agent fraction or polymeric carrier:anti-inflammatory agent fraction, respectively.

The chitosan-ASO-polymeric paste mixture (with or without a second pharmacologically active agent) can be drawn up into a syringe and injected through a needle (e.g., 18 gauge) directly into or onto (or proximal or close to) a localized target tissue or other target. The mixture then forms a semi-solid implant in the target tissue where the waxy polymer and chitosan protect the ASO therapeutic from degradation. Using an ASO therapeutic that is an anti-cancer agent, the chitosan and ASO therapeutic and polymeric paste mixture can be injected directly into a tumor (the tumor could even be fenestrated with the mixture). The polycationic polymer-negatively charged therapeutic system optionally incorporated into a polymeric carrier optionally with a second drug may be injected into the body to provide for a controlled release of one or more agents systemically.

Suitable polymeric carriers include, for example, biodegradable, nonbiodegradable and water soluble compositions. Representative examples of biodegradable compositions include albumin, gelatin, starch, cellulose, dextrans, polysaccharides, fibrinogen, polyesters such as poly(L-lactide), poly(D,L-lactide), poly(D,L-lactide-co-glycolide), poly(ε-caprolactone) and copolymers of the aforementioned polymers, polyglycolide, polyhydroxybutyrate, polyalkylcarbonate and polyorthoesters. See generally, Illum, L., Davids, S. S., (eds.) “Polymers in Controlled Drug Delivery” Wright, Bristol (1987); Arshady, J. Controlled Release 17:1-22 (1991); Pitt, Int'l. J. Pharmaceutics 59:173-196 (1990); Holland, et al., J. Controlled Release 4:155-180 (1986). Representative examples of nondegradable polymers include poly(ethylene-co-vinyl acetate), poly(ethylene-co-vinyl alcohol), urea based polyurethanes, polyurethanes, silicone rubber, polytetrafluoroethylene, polycarbonates, nylon polymer, polyethylene terephthalate, polyethylene and polymethylmethacrylate. Representative examples of water-soluble polymers include poly(ethylene glycol), polox, polyacrylic acid, poly(vinyl pyrrolidone), many polysaccharides and poly(vinyl alcohol).

Preferred polymeric carriers include polyethylene glycols, polyoxamers, polysaccharides, block copolymers of ethylene and propylene glycol such as poly(ethylene-co-vinyl acetate) (40% w/w ethylene and 60% w/w vinyl acetate), poly(D,L-lactide) oligomers and polymers, poly(L-lactide) oligomers and polymers, poly(glycolide), copolymers of lactic acid and glycolic acid, poly(ε-caprolactone), poly(valerolactone), polyanhydrides, copolymers of poly(ε-caprolactone) or poly(lactic acid) with poly(ethylene glycol), including all analogues, derivatives, conjugates and blends thereof.

Polymeric carriers can be fashioned in a variety of forms including, for example, microspheres, rod-shaped devices, pellets, slabs, capsules, films, pastes, gels, sprays, foams, and coatings or implantable medical devices. Goodell, et al., Am. J. Hosp. Pharm. 43:1454-1461 (1986); Langer, et al., Biomedical polymers, Polymericmaterials and Pharmaceuticals for Biomedical Use, Goldberg, E. P., Nakagim, A. (eds.) Academic Press, pp. 113-137 (1980); Rhine, et al., J. Pharm. Sci. 69:265:270 (1980); Brown, et al., J. Pharm. Sci. 72:1181-1185 (1983); Bawa, et al., J. Controlled Release 1:259-267 (1985). Anti-proliferative or anti-inflammatory agents may be dissolved in the polymer, suspended as particles, linked by occlusion in the matrices of the polymer, bound by covalent linkages, or encapsulated in microcapsules. The compositions can be provided in non-capsular formulations such as microspheres (ranging from nanometers to micrometers in size), pastes, threads of various sizes, films and sprays.

When the compositions are formed as a film. Such films are generally less than about 5, 4, 3, 2 or 1 mm thick, typically less than about 0.75 or 0.5 mm thick, and preferably less than about 500 to 25 μm thick. Such films are preferably flexible with a good tensile strength (for example, typically greater than about 50 N/cm², usually greater than about 100 N/cm², and preferably greater than about 150 or 200 N/cm²), have good adhesive properties (for example, to readily adhere to moist or wet surfaces) and have controlled permeability.

Controlling Release Rate.

The rate of negatively charged therapeutic release from the polycationic polymer, such as the rate of negatively charged ASO release from chitosan, can be controlled by modulating the ionic environment, such as the local phosphate ion concentration. For example, an increased concentration of anions, such as phosphate ions, may accelerate the release, whereas increased cations, such as ferric or calcium ions, may retards the rate of release. Other methods for modulating or controlling the release of products from the chitosan include: (1) entrapping the ASO-chitosan in a secondary polymeric matrix, discussed elsewhere herein, that reduces the diffusion rate of unbound (released) ASO from the system and slows release rates; (2) modulating the pH of the local area around the chitosan-ASO complex; and, (3) applying localized electric or magnetic fields around the localized chitosan injection site.

Such phosphate buffered saline (PBS) solutions or other phosphate-concentration-increasing compositions can be injected or otherwise administered to the area of the initial chitosan injection site. The compositions can also be administered by the inclusion of a phosphate-releasing compound in the injection area or by providing a systemic phosphate-concentration increasing composition.

Pharmacologically Active Agents.

The negatively charged therapeutic, called the first pharmacologically active agent, can be a negatively charged nucleic acid. The negatively charged nucleic acid can be a gene, oligonucleotide therapeutic, ASO, ribozyme, oligonucleotide RNA inhibitor, immune modulating oligonucleotide or other desired negatively charged nucleic acid. The first pharmacologically active agent can also be a negatively charged peptide or protein.

The second pharmacologically active agent can be a molecule that has an anti-proliferative and/or an anti-inflammatory pharmacological action.

The pharmacologically active agents can comprise anti-diabetic treatments, antimicrobial agents, anesthetics, vasoconstrictors, vasodilators, cardiotonics, enzymes, anti-inflammatories, hormones, bone metabolism controlling agents, hypotensives, sedatives, anti-cancer agents, antihistamines, antitussives, vaccines, anti-post surgical adhesion agents, anti-restenosis agents, anti-multiple sclerosis agents, anti-inflammatory bowel disease agents, and asthma treatments.

Methods of Administration.

The delivery systems and compositions, etc., can be administered orally, nasally, rectally, intravenously, intraperitoneally, intramuscularly, subcutaneously, intraarticularly, topically, directly or proximal or distal to the disease site, or otherwise as desired. The compositions can be localized at the disease site. For example, tumors can be treated by intra-tumoral injection of the composition. For example, tumors can be treated by peri-tumoral injection of the composition. For example, tumors can be treated by injection at a site distal to the tumor, where the agent(s) are delivery systemically.

The dose can be in the range of about 0.25 mg/m² to about 2000 mg m² of the nucleic acid or other negatively charged therapeutic. Other suitable ranges include from about 0.25 mg/m² to about 500 mg/m² of the nucleic acid or other negatively charged therapeutic and from about ² mg/m² to about 15 mg/m² of the nucleic acid or other negatively charged therapeutic.

Implantation Devices

A variety of surgical devices intended for implantation such as stents, sutures, indwelling catheters, prosthetics, and the like can be coated with or otherwise constructed to contain and/or release any of the anti-inflammatory agents provided herein. For example, stents comprise a generally tubular structure, and the surface is coated with at least one composition discussed herein. Thus, within some embodiments methods are provided for expanding the lumen of a body passageway, comprising inserting a stent into the passageway to effect such expansion, while simultaneously providing therapeutics. Examples of such passageways, and corresponding stents or other medical devices, include a biliary passageway, urethra, esophagus, and trachea-bronchus.

Representative examples of devices include cardiovascular devices (for example, implantable venous catheters, venous ports, tunneled venous catheters, chronic infusion lines or ports, including hepatic artery infusion catheters, pacemaker wires, implantable defibrillators); neurologic/neurosurgical devices (for example, ventricular peritoneal shunts, ventricular atrial shunts, nerve stimulator devices, dural patches and implants to prevent epidural fibrosis post-laminectomy, devices for continuous subarachnoid infusions); gastrointestinal devices (for example, chronic indwelling catheters, feeding tubes, portosystemic shunts, shunts for ascites, peritoneal implants for drug delivery peritoneal dialysis catheters, implantable meshes for hernias, suspensions or solid implants to prevent surgical adhesions, including meshes); genitourinary devices (for example, uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy); ophthalmic implants (for example, multino implants and other implants for neovascular glaucoma, drug eluting contact lenses for pterygium, splints for failed dacrocystalrhinostomy, drug eluting contact lenses for corneal neovascularity, implants for diabetic retinopathy, drug eluting contact lenses for high risk corneal transplants); otolaryngology devices (for example, ossicular implants and Eustachian tube splints or stents for glue ear or chronic otitis as an alternative to transtempanic drains); plastic surgery implants (for example, prevention of fibrous contracture in response to gel- or saline-containing breast implants in the subpectoral or subglandular approaches or post-mastectomy, or chin implants) and orthopedic implants (for example, cemented orthopedic prostheses).

Suitable stents include esophageal stents, gastrointestinal stents, vascular stents, biliary stents, colonic stents, pancreatic stents, ureteric and urethral stents, lacrimal stents, Eustachian tube stents, fallopian tube stents, nasal stents, sinus stents and tracheal/bronchial stents. Stents can be readily obtained from commercial sources or constructed in accordance with known techniques. Representative examples of stents include those discussed in U.S. Pat. Nos. 4,768,523; 4,776,337; 5,041,126; 5,052,998; 5,064,435; 5,089,606; 5,247,370; 5,176,626; and, 5,213,580; 5,328,47.

Venous access devices such as external tunneled catheters (for example, Hickman®/Broviac® and Groshong®), implanted ports, epidural catheters and peripherally inserted central catheters (PICCs), commonly used for prolonged venous access can comprise the compositions discussed herein. Infection, surgical adhesions and restenosis can be complications of access devices, Ascher, et al. (1993); Decker and Edwards (1998); Early, et al. (1990); Lam, et al. (1994); Press, et al. (1984); Raad, et al. (1993), Williams, et al. (1990). Thus the compositions discussed herein can also include an agent comprising antibiotic activity.

Additional Discussion of Certain Exemplary Diseases.

Cancers Generally: Cancer is the second leading cause of death in the U.S. and accounts for over 20% of all mortalities. Cancer is a proliferative disease and is characterized by the uncontrolled division of certain cells, which may lead to the formation of one or more tumors. A number of methods are used to treat cancer, including surgery, radiation, chemotherapy and combinations thereof. Although surgery is a relatively common method used for some localized tumors, there is still a significant chance of tumor recurrence after tumor excision.

Treating cancers and other proliferative diseases is limited by the potential for damage or toxicity to non-cancerous, healthy tissues. In radiation and surgical treatments, the procedure is generally confined to and proximal to the tumor sites. However, there can be significant risk to patients undergoing surgical removal of cancerous tissues (e.g., in removal of prostate or brain tumors there can be a significant risk of non-repairable damage to surrounding vital tissues, for example via potential reduced need for resection of non-tumor tissues. Furthermore, in focused radiation treatment, which is often given as a first line treatment for prostate cancer, there are similar risks. In the chemotherapeutic treatment of cancer, the drug is normally administered systemically, so that the whole body is exposed to the drug. These drugs are designed to be toxic to cancer cells, but they are also (generally) toxic to non-cancerous cells so that patients become quite ill when undergoing drug treatments for cancer. Through experience, oncologists are able to give doses of these drugs that may be tolerated by some patients. However, these doses are often not successful in treating cancers.

One major problem with any method of treating cancer is the local recurrence of the disease. For example, approximately 700,000 Americans are diagnosed with localized cancer annually (approximately 64% of all cancer patients) and almost half a million are treated using surgical methods. Unfortunately 32% of patients treated with surgery relapse after the initial treatment (approximately 21% relapse at the initial surgical site and 11% at distant metastatic sites). Almost 100,000 patients die annually due to localized recurrence of cancer. This is especially true in breast cancer where 39% of patients undergoing lumpectomy will experience local recurrence of the disease.

Staging is a method of judging the progress of the cancer (solid tumor) in a patient. A simplified approach puts patients into three groups or stages based on how far the cancer has advanced:

Stage 1: The cancer can be treated by surgically removing part of the organ. This is also known as the resectable stage.

Stage 2: The cancer has advanced past the point of being resectable, but is still confined to the organ itself.

Stage 3: The tumor has spread to other organs.

Many cancers are treated with anti-proliferative agents including, for example, 5-fluorouracil (Efudex®), vinca alkaloids (for example, vincristine (Oncovin®)), anthracyclines (for example, doxorubicin (Adriamycin®)), cisplatin (Platinol-AQ®), gemcitabine hydrochloride (Gemzar®), methotrexate and paclitaxel. Some examples of the toxicities associated with the anti-proliferative agents, methotrexate and paclitaxel, are discussed elsewhere herein. Methotrexate is used to treat several cancers including, for example, bladder, breast, cervical, head and neck, hepatic, lung, and testicular cancers. Paclitaxel is used to treat several cancers including, for example, ovarian, breast, and non-small cell lung cancers. Compendium of Pharmaceutical and Specialties Thirty-fifth Edition (2000).

Toxicities due to 5-fluorouracil can include cardiovascular toxicity such as myocardial ischemia; central nervous system toxicities such as euphoria, acute cerebellar syndrome and ataxia; dermatologic toxicities such as alopecia and dermatitis; gastrointestinal toxicities such as nausea, vomiting and oral or gastrointestinal ulceration; hematologic toxicities such as leukopenia, thrombocytopenia and anemia; hypersensitivity toxicities such as anaphylaxis and contact hypersensitivity; ocular toxicities such as increased lacrimation, photophobia and conjunctivitis; and, other toxicities such as fever. 5-fluorouracil is used to treat many cancers including, for example, breast, colorectal, gastric, hepatic, bladder, head and neck, non-small cell lung, ovarian, pancreatic, and prostate cancers. Compendium of Pharmaceutical and Specialties Thirty-fifth Edition (2000).

Toxicities due to vincristine include central nervous system toxicities such as seizures in children and hallucinations; dermatoligic toxicity such as alopecia; extravasation toxicity such as vesicant; gastrointestinal toxicities such as nausea, vomiting, constipation and stomatitis; hematologic toxicity such as myelosuppression; neurologic toxicities such as peripheral neuropathy and autonomic neurophathy; ocular toxicities such as double vision, transient blindness and optic atrophy; renal/metabolic toxicities such as urinary retention, hyperuricemia and bladder atony; respiratory toxicity such as shortness of breath; and, other toxicity such as fever in children. This anti-proliferative agent is used to treat several cancers including, for example, Hodgkin's disease, small cell lung, Wilm's tumor, and testicular cancers. Compendium of Pharmaceutical and Specialties Thirty-fifth Edition (2000)

Toxicities due to doxorubicin include cardiovascular toxicities such as electrocardiographic abnormalities and cardiomyopathy; dermatologic toxicities such as alopecia and nail changes; extravasation hazard toxicity such as vesicant; gastrointestinal toxicities such and nausea, vomiting and stomatitis; genitourinary toxicity such as red coloration of urine; hematologic toxicity such as myelosuppression; hypersensitivity toxicities such as anaphylaxis and skin rash; ocular toxicity such as conjunctivitis; reproductive toxicity such as infertility; and, other toxicity such as hyperuricemia. This anti-proliferative agent is used to treat several cancer including, for example, breast, small cell lung, and ovarian cancers. Compendium of Pharmaceutical and Specialties Thirty-fifth Edition (2000)

Toxicities due to cisplatin include cardiovascular toxicity such as electocardiographic changes; dermatologic toxicity such as hyperpigmentation; extravasation hazard toxicity such as irritant; gastrointestinal toxicities such as nausea and vomiting; hematologic toxicities such as myelosuppression and hemolytic anemia; hypersensitivity toxicity such as anaphylactic; neuromuscular toxicity such as peripheral neurophathy and acute encephalopathy; ocular toxicity such as retrobulbar neuritis; otologic toxicities such as hearing loss and tinnitus; renal/metabolic toxicities such as toxic nephropathy and hypokalemia; and, other toxicity such as infertility. This anti-proliferative agent is used to treat several cancers including, for example, bladder, small cell lung, ovarian, testicular, brain, breast, cervical, head and neck, hepatoblastoma, and thyroid cancers. Compendium of Pharmaceutical and Specialties Thirty-fifth Edition (2000)

Toxicities due to gemcitabine hydrochoride include, for example, hematologic toxicities such as myelosuppression; gastrointestinal toxicities such as nausea, vomiting and somatitis; hepatic toxicities such as transient elevations of serum transaminases; renal toxicities such as proteinuria, hematuria, hemolytic uremicsyndrome and renal failure; dermatologic toxicity such as rash and alopeica; edema toxicities such as edema and peripheral edema; and, other toxicity such as fever. This anti-proliferative agent is used to treat pancreatic and non-small cell lung cancers. Compendium of Pharmaceutical and Specialties Thirty-fifth Edition (2000)

The present invention comprises prevention or treatment of localized cancers or solid tumors that can be treated include those of the prostate, breast, pancreas, liver, kidney, genitourinary system, brain, gastrointestinal system, respiratory system, and head and neck. The invention may prevent or treat cancers, including metastases, by allowing controlled release of drugs at a site somewhat distant from the target tumors by allowing effective concentrations of the drug(s) to reach the tumors and/or metastases by diffusion or even systemic transport. Some of these cancers are discussed further in the following paragraphs.

Prostate Cancer: Prostate cancer is a malignant tumor that arises in the cells lining the prostate gland. In the U.S., an estimated 200,000 patients will develop prostate cancer this year, and more than 30,000 will die of the disease. Prostate cancer has a deaths to new cases ratio of ˜15%. The cancer may remain within the prostate, or it may spread to surrounding tissues or to distant sites (most often lymph nodes and bone). Usually prostate cancer spreads silently, producing symptoms only when it has progressed beyond the prostate. If prostate cancer is diagnosed and treated during early stages, patients have a 5-year survival rate of 94%.

Prostate cancer is often discussed as a disease of men over age 50. In fact, 80% of men with prostate cancer are 60 years of age and older. A man's chances of being diagnosed with prostate cancer during his lifetime are about 1 in 10, roughly the same as a woman's chances of having breast cancer. The number of reported new cases has risen dramatically in recent years as a result of improved tests that can detect the disease early in its development, often long before symptoms appear. The likelihood of developing prostate cancer in any given year increases with age, but rises dramatically after age 50.

Current treatment options for prostate cancer depend upon the extent of disease progression, the patient's age and overall health. Elderly patients, who have only early stage cancer or who suffer from additional, more serious diseases, may be treated conservatively, whereas those whose cancer is advanced may undergo more aggressive treatment. Prostate cancer is currently treated by various methods, including radiation therapy (external beam radiation or brachytherapy), hormone withdrawal or castration (surgical or chemical), anti-proliferative agents, surgery, and expectant therapy (that is, “watchful waiting”). No treatment guarantees an absolute cure, and some have considerable side effects.

Early stage prostate cancer (that is, the tumor is localized to the prostate) may be treated with “watchful waiting”. Surgery for prostate cancer is often recommended for patients whose overall health is otherwise good and the tumor is confined to the prostate gland. A common treatment for localized cancer of the prostate in men under the age of 70 is radical prostatectomy (that is, surgical removal of the prostate).

Patients whose cancer is localized in the prostate area are commonly treated with external beam radiation (EBR). The radiation kills cancer cells and shrinks tumors. EBR accounts for less than 20% of localized prostate cancer treatment, with approximately 50% of these patients experiencing post radiation recurrences of the disease. Combined with early stage prostate cancer detection and increased demand from patients, brachytherapy (i.e., local radiation therapy) use is expected to grow. In 1995, only 2.5% of newly diagnosed patients were treated using brachytherapy. Brachytherapy involves the implantation of radioactive metal “seeds” in the prostate tumor.

Treatment for prostate cancer that has spread involves removal of the testicles or hormone therapy. Both are used to inhibit or stop the production of the testosterone that is driving the cancer growth. Approximately 20% of all prostate cancer patients undergo hormone withdrawal therapy. Hormone therapies include goserelin acetate (Zoladex®) or leuprolide acetate (Lupron®). Anti-proliferative agents used to treat prostate cancer include 5-fluorouracil. Paclitaxel is currently undergoing clinical trials for use against prostate cancer and 5-fluorouracil is used to treat prostate cancer.

Breast Cancer: In the U.S., breast cancer is the most common cancer among women, with about 180,000 new cases diagnosed every year (male breast cancer accounts for about 5% of all diagnosed breast cancers). It is surpassed only by lung cancer as a cause of death in women, and it is responsible for approximately 50,000 deaths annually. An American woman has a one in eight (or about 13%) chance of developing breast cancer during her lifetime. Over the past decade, most reported breast cancers were small, primary (arising independently; not caused by a metastasis) tumors. Roughly 70% to 80% of newly diagnosed patients exhibited early-stage disease (Stage 1 or 2), and a majority had no involvement of the axillary (underarm) lymph nodes.

Most breast cancers are carcinomas (that is, malignant tumors that grow out of epithelial tissues). Less than 1% of breast cancers are sarcomas, or tumors arising from connective tissue, bone, muscle or fat. In addition, most breast cancers (about 75%) are ductal carcinomas, arising in the tissues that line the milk ducts. A much smaller number of cancers (about 7%) are found within the breast lobules and are called lobular carcinomas. Paget's disease (cancer of the areola and nipple) and inflammatory carcinoma account for nearly all other forms of breast cancer.

Breast cancer treatment is complicated and depends on many factors. Two important factors are the type of tumor and the stage of progression. Tumor characteristics, in particular, help to separate individuals into two groups: (1) those who are at low risk of cancer recurrence and (2) those who are at high risk of cancer recurrence. Specific prognostic factors place patients in either of these groups. These factors include tumor size; presence of female sex hormone estrogen and progesterone (ER/PR) receptors; cellular growth cycle phase (whether tumor cells are actively dividing or are in “S-phase”); presence of a protein known as “her-2-neu protein”; tumor grade, an indicator of tumor cell differentiation or change; and, tumor ploidy, the number of sets of genetic material within tumor cells.

Treatment of primary disease without significant lymph node involvement is by lumpectomy and radiotherapy. More significant lymph node involvement may warrant mastectomy and removal of auxiliary lymph nodes. At this stage the chance of metastasis and local recurrence is high. Treatment of metastatic disease is palliative, involving radiation therapy and chemotherapy, which are immunosuppressive, cytotoxic and leukopoenic. Anti-proliferative agents including, for example, 5-fluorouracil, doxorubicin, methotrexate, and paclitaxel, have been approved for use against breast cancer.

Pancreatic Cancer: The pancreas is an organ of the digestive system located near the stomach and small intestine. It has two major functions: the production of enzymes and hormones. Cancers of the pancreas can occur in the exocrine (i.e., enzymes) pancreas (e.g., classic pancreatic adenocarcinomas) or can occur in the endocrine (i.e., hormones) pancreas.

Cancers of the exocrine pancreas are a very serious health issue. In the U.S., approximately 28,000 patients are diagnosed with pancreatic cancer, while about the same number die annually from this disease. Pancreatic cancer occurs equally in males and females. Due to difficulties in diagnosis, the intrinsic aggressive nature of pancreatic cancers, and the sparse systemic treatment options available, only approximately 4% of patients diagnosed with pancreatic adenocarcinoma live for 5 years after diagnosis. Pancreatic cancer is the 5^{th} leading cause of cancer death, following breast, lung, colon, and prostate cancer.

The choice of treatment for pancreatic cancer depends largely on the stage of the tumor. Possible treatments include surgery, anti-proliferative agents, radiation, and biological therapy. Surgery is usually reserved for Stage 1 patients whose cancer is deemed resectable. Sometimes a combination of therapies, such as radiation and anti-proliferative agent given before or after surgery, can increase a patient's chances of survival. Pancreatic cancer that is deemed unresectable (usually Stage II or later) may be treated using anti-proliferative agents in clinical trials. Anti-proliferative agents, such as, for example, gemcitabine or 5-fluorouracil have had some effect against pancreatic cancer and gemcitabine is used as a palliative agent. Toxicities due to these anti-proliferative agents are discussed elsewhere herein. Radiation therapy has some effect against pancreatic cancer when used in combination with chemotherapy. Radiation therapy alone may subdue symptoms. This form of treatment is also used in Stage II or later pancreatic cancers.

Bladder Cancer: In 1998, it was estimated that over 54,000 new cases of bladder cancer would be diagnosed in the U.S. and about 15,000 deaths would be attributed to the disease. Bladder cancer is now the fourth most common cancer among American men and the ninth most common cancer among American women. It occurs three times more frequently in men than in women. Primarily a disease of older men, bladder cancer is a significant cause of illness and death. The risk of bladder cancer increases steeply with age (80% of cases occur in people older than 50 years), with over half of all bladder cancer deaths occurring after age 70. In white men over 65, the annual disease rate of bladder cancer is approximately 2 cases per 1,000 persons; this contrasts with a rate of 0.1 cases per 1,000 persons under 65. During one's lifetime, the probability of developing bladder cancer is greater than 3%; however, the probability of dying, from bladder cancer is small (<1%). Bladder cancer rarely occurs in people who are younger than 40 years of age.

Recent studies suggest that certain genes and inherited metabolic abilities may play a role in bladder cancer. Transitional cell carcinoma (TCC) is the most common form of bladder cancer. TCC usually occurs as a superficial (surface), papillary (wart-like), exophytic (outward-growing) mass upon a stalk-like base. In some cases, though, TCC may be attached on a broad base or it may appear ulcerated (within an indented lesion). Papillary TCCs often start out as areas of hyperplasia that later dedifferentiate, or lose individual cell characteristics. Only about 10% to 30% of papillary TCCs develop into invasive cancers. By contrast, nonpapillary forms of TCC are more likely to become invasive. As noted, such TCCs may appear ulcerated or flat. Flat, nonpapillary TCC that is made up of anaplastic epithelium is classified as carcinoma in situ (CIS or TIS). The tissue of CIS contains cells that are large, have noticeable nucleoli (round body within a cell; involved in protein synthesis), and lack normal polarity.

The treatment of bladder cancer depends upon many factors. The most important of these factors are the type of tumor that is present and its stage. Common treatments include transurethral resection (TUR), electrosurgery, laser surgery, intravesical therapy, anti-proliferative agents, surgical therapy, cystectomy, and radiation therapy. Examples of anti-proliferative agents used to treat bladder cancer include, for example, 5-fluorouracil, cisplatin and methotrexate. Toxicities due to the anti-proliferative agents, 5-fluorouracil, cisplatin, and methotrexate, are discussed elsewhere herein.

Brain Cancer: Brain tumors are often inoperable and more than 80% of patients die within 12 months of diagnosis. Approximately 18,000 new cases of primary intracranial (brain) cancer are diagnosed each year in the U.S. This represents about 2 percent of all adult cancers. More than 50 percent of these are high-grade gliomas (i.e., glioblastoma multiform and anaplastic astrocytoma tumors). Patients with these tumors often suffer from severe disabilities such as motor dysfunction, seizures, and vision abnormalities.

Tumors that begin in brain tissue are known as primary brain tumors. Primary brain tumors are classified by the type of tissue in which they begin. The most common brain tumors are gliomas, which begin in the glial (supportive) tissue. Others include astrocytomas, brain stem gliomas, ependymomas and oligodendrogliomas.

Surgical removal of brain tumors is recommended for most types and in most locations and should be as complete as possible within the constraints of preservation of neurologic function. An exception to this rule is for deep-seated tumors, such as pontine gliomas, which are diagnosed on clinical evidence and are treated without initial surgery approximately 50% of the time. In the majority of cases, however, diagnosis by biopsy is preferred. Stereotaxic biopsy can be used for lesions that are difficult to reach and resect. Patients who have brain tumors that are either infrequently curable or unresectable should be considered candidates for clinical trials that evaluate radiosensitizers, hyperthermia, or interstitial brachytherapy used in conjunction with external-beam radiation therapy to improve local control of the tumor or for studies that evaluate new drugs and biological response modifiers.

Radiation therapy has a major role in the treatment of most tumor types and can increase the cure rate or prolong disease-free survival. Radiation therapy may also be useful in the treatment of recurrences in patients treated initially with surgery alone. Chemotherapy may be used before, during, or after surgery and radiation therapy. Recurrent tumors are treated with chemotherapy as well. Anti-proliferative agents used in the treatment of brain cancers include cisplatin. Examples of the toxicities associated with this anti-proliferative agent are discussed elsewhere herein.

Restenosis

Restenosis is a form of chronic vascular injury leading to vessel wall thickening and loss of blood flow to the tissue supplied by the blood vessel. This inflammatory disease can occur in response to vascular reconstructive procedures including any manipulation that relieves vessel obstruction. Thus restenosis is a major restrictive factor limiting the effectiveness of these procedures. At present, there are no approved treatments for the prevention of restenosis in humans. Clinical trials are currently ongoing using paclitaxel (Taxol™) to treat or prevent this disease.

The present invention comprises prevention or treatment of restenosis, for example by administering to a blood vessel a therapeutically effective amount of the combination of an oligonucleotide therapeutic and an anti-inflammatory agent. Suitable compositions include a polymeric carrier that can be surgically implanted at a restenosis site, or potential restenosis site, or can be injected via a catheter as a polymeric paste or gel.

Arthritis

Rheumatoid arthritis (RA) is a debilitating chronic inflammatory disease characterized by pain, swelling, synovial cell proliferation (pannus formation) and destruction of joint tissue. In the advanced stage, the disease often damages critical organs and may be fatal. The disease involves multiple members of the immune system (macrophages/monocytes, neutrophils, B cells and T cells) complex cytokine interactions and synovial cell malfunction and proliferation. Early aggressive treatment is now recommended with disease modifying anti-rheumatic drugs (DMARDs) such as methotrexate, which drug is discussed elsewhere herein.

Crystal induced arthritis is characterized by crystal induced activation of macrophages and neutrophils in the joints and is followed by excruciating pain for many days. The disease progresses so that the intervals between episodes gets shorter and morbidity for the patient increases. This disease is generally treated symptomatically with non-steroidal anti-inflammatory drugs (NSAIDs) such as diclofenac sodium (Voltaren®). This anti-inflammatory agent has toxicities which include central nervous system toxicities such as dizziness and headache; dermatologic toxicities such as rash and pruritus; gastrointestinal toxicities such as exacerbated ulcerative colitis and Crohn's disease; genitourinary toxicities such as acute renal failure and renal papillary necrosis; hematologic toxicities such as agranulocytosis, leukopenia and thrombocytopenia; hepatic toxicities such as elevated liver transaminases and hepatitis; and, other toxicities such as asthma and anaphylaxis.

The methods herein, etc., prevent, treat or inhibit (similar to the effects on certain other diseases herein) rheumatoid arthritis, for example via administering to a patient a therapeutically effective amount of an oligonucleotide therapeutic and optionally an anti-inflammatory agent. Suitable compositions include a polymeric carrier that can be injected into a joint as a controlled release carrier of the anti-inflammatory agent and microparticulates as controlled release carriers of the oligonucleotide therapeutic (which in turn has been incorporated in the polymeric carrier). Such polymeric carriers may take the form of polymeric microspheres, pastes or gels.

Surgical Adhesions

Surgical adhesion is a complex inflammatory disease in which tissues that normally remain separated in the body grow into each other, usually as a result of surgical trauma. These adhesions, including adhesions from other causes, are a major cause of failed surgical procedures, bowel obstruction and infertility. Other adhesion-related complications include chronic pelvic pain, urethral obstruction and voiding dysfunction. Inflammatory processes include neutrophil accumulation and activation in the traumatized tissues, fibrin deposition and bonding of adjacent tissues, macrophage invasion, fibroblast proliferation into the area, collagen deposition, angiogenesis and the establishment of permanent adhesion tissues. Current therapies include the use of steroidal and non-steroidal anti-inflammatory drugs (examples of toxicities from these types of agents are discussed elsewhere herein).

The compositions, etc., herein inhibit or treat surgical adhesions, for example, by administering an oligonucleotide therapeutic and optionally an anti-inflammatory agent. The oligonucleotide therapeutic is optionally associated with microparticulates and can be administered directly to the surgical site.

Inflammatory Conditions

The compositions, etc., herein may optionally inhibit or treat inflammatory conditions involving neutrophils for example comprising administering to a patient compositions containing an oligonucleotide therapeutic and an anti-inflammatory agent. Examples of such conditions include crystal-induced arthritis; osteoarthritis; non-rheumatoid inflammatory arthritis; mixed connective tissue disease; Sjögren's syndrome; ankylosing spondylitis; Behcet's syndrome; sarcoidosis; psoriasis; eczema; inflammatory bowel disease; chronic inflammatory lung disease; neurological disorders; and, multiple sclerosis. Some of these diseases are discussed further in the following paragraphs.

Inflammatory bowel disease (IBD): This disease refers mainly to Crohn's disease and ulcerative colitis that affect the intestine. IBD is an inflammatory disease characterized by periods of flare and remission. Joint inflammation may occur at the same time as a flare of IBD. Other complications of IBD may include inflammation of the skin, mouth, eye and may lead to cancer of the intestine. Chronic symptoms of this disease include intestinal blockage, perforation, abscess and bleeding. Symptoms may be treated with non-steroidal anti-inflammatory agents such as 5-aminosalicylic acid (Salofalk®). This anti-inflammatory agent has toxicities which include cardiovascular toxicity such as myocarditis; central nervous system toxicities such as headache and dizziness; gastrointestinal toxicities such as nausea and vomiting and diarrhea; genitourinary toxicities such as nephrotic syndrome and interstitial nephritis; hypersensitivity toxicities such as rash and pruritis; neuromuscular toxicity such as neuropathy; and, other toxicities such as hair loss and lichen planus.

Chronic inflammatory lung diseases: These inflammatory diseases include asthma, pneumoconiosis, obstructive pulmonary disease, nasal polyps and pulmonary fibrosis. Typically, such diseases are characterized by immune cell (such as neutrophils, macrophages and lymphocytes) activation and invasive inflammatory processes and thickening of the affected masses. Current drug therapies include the use of steroidal anti-inflammatory agents such as prednisone (Deltasone®). This anti-inflammatory agent has toxicities which include cardiovascular toxicities such as sodium and water retention; central nervous system toxicities such as headache, depression and convulsions; dermatologic toxicities such as impaired wound healing and acne; endocrine/metabolic toxicities such as menstrual irregularities and hypothalamic-pituitary-adrenal (HPA) axis suppression, Cushingoid appearance (e.g., moon faces, central obesity), growth suppression in children and osteoporosis; gastrointestinal toxicities such as peptic ulcer and pancreatitis; neuromuscular toxicity such as myopathy; ocular toxicities such as posterior subcapsular cataracts and glaucoma; and, other toxicities such as aseptic necrosis of femoral and humeral heads, spontaneous fractures and increased infection risk.

Chronic inflammatory skin diseases (including psoriasis and eczema): Psoriasis is a common, chronic inflammatory skin disease characterized by raised, thickened and scaly lesions which itch, burn, sting and bleed easily. While these diseases have cellular proliferation and angiogenic components in later stages of the disease, patients often have accompanying arthritic conditions. Symptoms may be treated with steroidal anti-inflammatory agents such as prednisone or anti-proliferative agents such as methotrexate, which agents are discussed elsewhere herein.

The following provides some additional representative examples of inflammatory diseases that can be treated, etc., include, for example, arterial embolization in arteriovenous malformations (vascular malformations); menorrhagia; acute bleeding; central nervous system disorders; and, hypersplenism; inflammatory skin diseases such as psoriasis; eczematous disease (atopic dermatitis, contact dermatitis, eczema); immunobullous disease; and, inflammatory arthritis which includes a variety of conditions including rheumatoid arthritis, mixed connective tissue disease, Sjögren's syndrome, ankylosing spondylitis, Behcet's syndrome, sarcoidosis, crystal induced arthritis and osteoarthritis (all of which feature inflamed, painful joints as a prominent symptom).

Further representative diseases include inflammatory bowel disease (IBD) including ulcerative colitis and Crohn's disease; surgical adhesions; periodeontal disease; polycystic kidney disease; chronic inflammatory diseases of the respiratory tract including asthma, chronic obstructive pulmonary disease (COPD), chronic bronchitis, asthmatic bronchitis, chronic obstructive bronchitis, and emphysema and other diseases which lead to chronic airway obstruction; diseases associated with the obstruction of body passageways including, for example, vascular diseases, neoplastic obstructions, inflammatory diseases and infectious diseases; and, neovascular diseases of the eye including, for example, corneal neovascularization, neovascular glaucoma, proliferative diabetic retinopathy, retrolental fibroblasia and macular degeneration.

The compositions discussed herein can also be used to treat vascular diseases that cause obstruction of the vascular system. Such diseases include artherosclerosis of all vessels (around any artery, vein or graft) including, but not restricted to: the coronary arteries, aorta, iliac arteries, carotid arteries, common femoral arteries, superficial femoral arteries, popliteal arteries, and at the site of graft anastomosis; vasospasms (for example, coronary vasospasms and Raynaud's disease); restensosis (obstruction of a vessel at the site of a previous intervention such as balloon angioplasty, bypass surgery, stent insertion and graft insertion); inflammatory and autoimmune conditions (for example, temporal arteritis and vasculitis).

The compositions can be used for preventing or treating inflammatory diseases, acute or chronic, which affect or cause the obstruction of a body passageway. Representative examples include vasculitis (for example, giant cell arteritis (temporal arteritis and Takayasu's arteritis), polyarteritis nodosa, allergic angiitis and granulomatosis (Churg-Strauss disease), polyangiitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis; gastrointestinal tract diseases (for example, pancreatitis, Crohn's disease, ulcerative colitis, ulcerative proctitis, primary sclerosing cholangitis, benign strictures of any cause including idiopathic (for example, strictures of bile ducts, esophagus, duodenum, small bowel or colon)); respiratory tract diseases (for example, asthma, hypersensitivity pneumonitis, asbestosis, silicosis and other forms of pneumoconiosis, chronic bronchitis and chronic obstructive airway disease); nasolacrimal duct diseases (for example, strictures of all cases including ideopathic); and, Eustachian tube diseases (for example, strictures of all cases including ideopathic).

The compositions can also be used for treating or preventing infectious diseases associated with or causative of the obstruction of a body passageway. Briefly, infectious diseases include several acute and chronic infectious processes that can result in obstruction of body passageways including, for example, obstructions of the male reproductive tract (for example, strictures due to urethritis, epididymitis, prostatitis); obstructions of the female reproductive tract (for example, vaginitis, cervicitis, pelvic inflammatory disease (for example, tuberculosis, gonococcus, chlamydia, enterococcus and syphilis)); urinary tract obstructions (for example, cyctitis, urethritis); respiratory tract obstructions (for example, chronic bronchitis, tuberculosis, other mycobacterial infections (MAI, etc.), anaerobic infections, fungal infections and parasitic infections); and, cardiovascular obstructions (for example, mycoticaneurysms and infective endocarditis).

Pharmaceutical Products

The invention also provides pharmaceutical products, comprising compositions as discussed herein in a container. The products can also include a notice associated with the container, typically in a form prescribed by a governing agency regulating the manufacture, use, or sale of pharmaceuticals or biopharmaceuticals, whereby the notice is reflective of approval by the agency of the compositions, such as an oligonucleotide therapeutic and an anti-proliferative agent or anti-inflammatory agent, for human or veterinary administration to treat proliferative diseases or inflammatory diseases (such as, for example, inflammatory arthritis, restenosis, surgical adhesions, psoriasis, graft rejections, inflammatory bowel disease, multiple sclerosis, and inflammatory lung disease). Instructions for the use of the agents or composition may also be included. Such instructions may include information relating to the dosing of a patient and the mode of administration.

### EXAMPLES

### Example 1

Preparation of Various Compositions.

Preparation of chitosan-ASO therapeutic particles. 28 mg of sodium chloride was added to 72 mg of medical grade chitosan (Carbomer, Inc., Westborough, Mass.). This mixture was placed in a ball and mill pulverizer for 15 minutes to reduce the particle size to approximately 1-30 μm. This pulverized mixture was placed in a 20 ml glass vial. Thirty-six milligrams of negatively charged Clusterin ASO (an ASO agent shown to inhibit the production of clusterin protein (a pro-survival protein)) with a phosphorothioate backbone was dissolved in 500 μi of distilled water. This ASO solution was added to the chitosan in the glass vial and the contents were allowed to dry overnight at 37° C.

Preparation of polymeric paste. Into a 20 ml glass vial were placed 600 mg of the liquid polymer methoxypolyethylene glycol 350 (Union Carbide, Danbury, Conn.) followed by 400 mg of solid/waxy biodegradable triblock polymer of poly(DL-lactide-co-caprolactone) (PLC) and poly(ethylene glycol) (PEG) (with a final triblock copolymer structure of PLC-PEG-PLC, abbreviated as TB). These were blended into the polymer dispersion using a spatula and gentle heat at 40° C. (water bath).

Preparation of final microparticulate in paste. 40 mg of the chitosan/oligonucleotide microparticulate was added to the 1000 mg of paste. The mixture was blended into a homogenous dispersion using a spatula and warming at 40° C. for 15 minutes. The warm blend was then immediately sucked up into a 1 ml plastic syringe using an 18 gauge needle. The formulation was then stored at 4° C. until use.

### Example 2

The Effect of Clusterin Antisense Complexed to Chitosan Microparticles and Incorporated Into a Polymeric Paste Loaded With Paclitaxel on PC-3 Human Prostate Tumors in SCID Mice.

An in vivo study was carried out using a medicament manufactured as follows: Negatively charged phosphorothioated clusterin ASO or negatively charged phosphorothioated control oligonucleotide was initially complexed to a chitosan microparticulate compartment to form a microparticulate:oligonucleotide therapeutic fraction. Paclitaxel (an anti-proliferative agent) was dissolved or suspended in appropriate pastes by physical blending in a paste of a biodegradable triblock polymer of poly(DL-lactide-co-caprolactone) (PLC) and poly(ethylene glycol) (PEG) (with a final triblock copolymer structure of PLC-PEG-PLC, abbreviated as TB) blended with low molecular weight liquid methoxy-poly(ethylene glycol) (MePEG) in a ratio of 40:60 TB:MePEG to optionally form a polymeric carrier:anti-proliferative agent fraction. The microparticulate:oligonucleotide therapeutic fraction was then dispersed in the polymeric carrier:anti-proliferative agent fraction by physical blending to form a homogenous paste.

Six week old SCID mice were inoculated subcutaneously in the flank region with 1×10⁶ PC-3 human prostate cancer cells and 0.1 ml Matrigel while the mice were under methoxyflurane anesthesia. When the tumors reached approximately 1 cm³ in size the mice were randomly assigned to one of three different paste groups: 1) Chitosan complexed with Antisense (phosphorothioated clusterin ASO), incorporated into Paste (TB:MePEG paste) (that is, Chitosan+Antisense+Paste), 2) Chitosan complexed with Control Antisense (Control Antisense is a mismatch oligonucleotide, also abbreviated as MM or MM-ASO), incorporated into Paste that also contains Paclitaxel (that is, Chitosan+Control Antisense+Paste+Paclitaxel), and 3) Chitosan complexed with Antisense, incorporated into Paste that also contains Paclitaxel (that is, Chitosan+Antisense+Paste+Paclitaxel). Chitosan had a final loading in the pastes of 4% w/w, Control Antisense had a final loading in the appropriate pastes of 2% w/w, Antisense had a final loading in the appropriate pastes of 2% w/w, and Paclitaxel had a final loading in the appropriate pastes of 1% w/w. One hundred milligrams paste from the appropriate group was then injected into each tumor. Each group started with 6 mice. Tumor volume was measured once weekly and calculated using the formula: length×width×height×0.5236.

The results, as shown in FIG. 1, demonstrated that based on tumor volume the Chitosan+Antisense+Paste+Paclitaxel treatment resulted in tumor regression or inhibition of tumor growth for approximately 5 weeks. Each datum point represents the mean of results for a minimum of 4 mice (if more than 2 mice died in each group, then the data are not shown). Each error bar represents the standard deviation for its respective datum point.

Previous work using only the TB:MePEG paste loaded only with paclitaxel (containing no chitosan and containing no clusterin ASO) required a paclitaxel loading of 10% w/w, Jackson, J. K., et al., Cancer Res. 60:4146-4151 (2000), to achieve similar efficacy to the clusterin ASO-chitosan-1% paclitaxel paste in this Example. The prior work also resulted in greater toxicities compared to this study. Thus, the present invention treated a proliferative disease using less anti-proliferative agent than was required previously and decreased the side effects or toxicities.

Previous work using intraperitoneal injections of phosphorothioated clusterin ASO and intravenous injections of paclitaxel required daily administration of the clusterin ASO for approximately two weeks followed by daily administration of the paclitaxel for approximately three weeks. Miyake, H., et al., Clin. Cancer Res. 6:1655-1663 (2000). Surprisingly, the present invention used less clusterin ASO and less paclitaxel to achieve approximately the same efficacy as the previous work. Thus, the present invention used less oligonucleotide therapeutic, anti-proliferative agent, and injections to achieve approximately the same efficacy as the protocol in the previous work. This also showed a decrease in the elimination and degradation of the oligonucleotide therapeutic.

### Example 3

The Effect of Clusterin Antisense Complexed to Chitosan Microparticles and Incorporated Into a Polymeric Paste Loaded With Paclitaxel On LNCaP Human Prostate Tumors in SCID Mice.

An in vivo study was carried out using medicaments prepared as in Example 1. Six week old SCID mice were inoculated subcutaneously in the flank region with 1×10⁶ LNCaP human prostate cancer cells and 0.1 ml Matrigel while the mice were under methoxyflurane anesthesia. Blood samples were obtained using tail vein incisions and prostate specific antigen (PSA) levels were determined weekly with an enzymatic immunoassay kit according to the manufacturer's protocol. (PSA is used as an endpoint for androgen independence progression of prostate tumors.) The mice were castrated when their serum PSA level rose above 50 ng/ml. After castration the serum PSA levels decreased. When the serum PSA levels increased to greater than 60 ng/ml the mice were randomly assigned to one of three different paste groups: 1) Chitosan complexed with Antisense (phosphorothioated clusterin ASO), incorporated into Paste (TB:MePEG paste) (that is, Chitosan+Antisense+Paste), 2) Chitosan complexed with Control Antisense (Control Antisense is a mismatch oligonucleotide, also abbreviated as MM or MM-ASO), incorporated into Paste that also contains Paclitaxel (that is, Chitosan+Control Antisense+Paste+Paclitaxel), and 3) Chitosan complexed with Antisense, incorporated into Paste that also contains Paclitaxel (that is, Chitosan+Antisense+Paste+Paclitaxel). Chitosan had a final loading in the pastes of 4% w/w, Control Antisense had a final loading in the appropriate pastes of 2% w/w, Antisense had a final loading in the appropriate pastes of 2% w/w, and Paclitaxel had a final loading in the appropriate pastes of 1% w/w. One hundred milligrams paste from the appropriate group was then injected into each tumor. Each group started with 5-6 mice. Tumor volume was measured once weekly and calculated using the formula: length×width×height×0.5236.

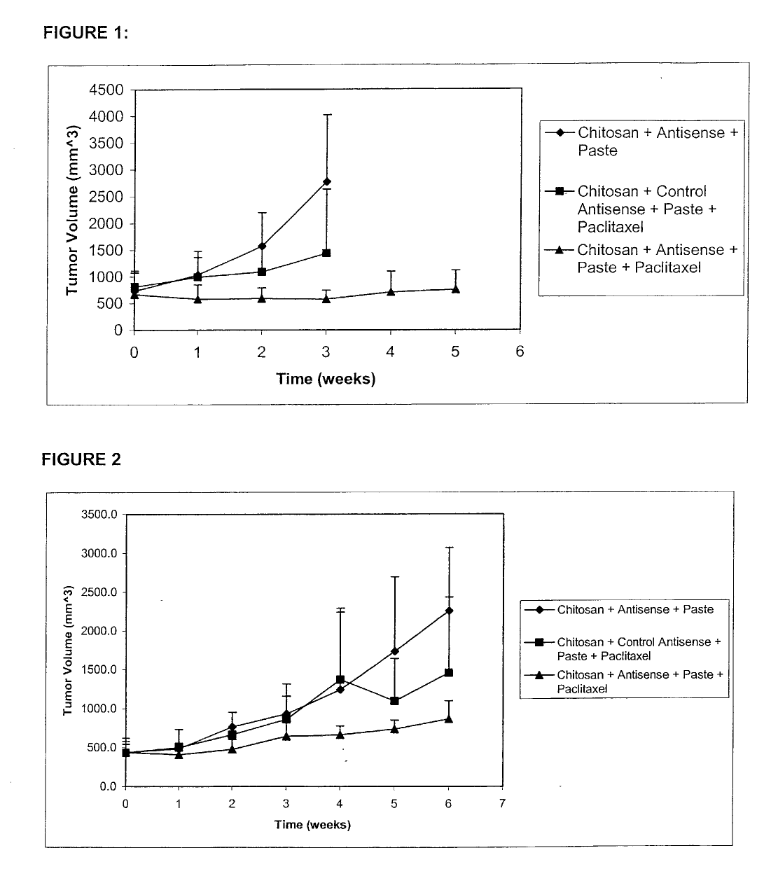

The results, as shown in FIG. 2, demonstrated that based on tumor volume the Chitosan+Antisense+Paste+Paclitaxel treatment resulted in tumor regression or inhibition of tumor growth for approximately 6 weeks. Each datum point represents the mean of results for a minimum of 3 mice (if more than 2-3 mice died in each group, then the data are not shown). Each error bar represents the standard deviation for its respective datum point.

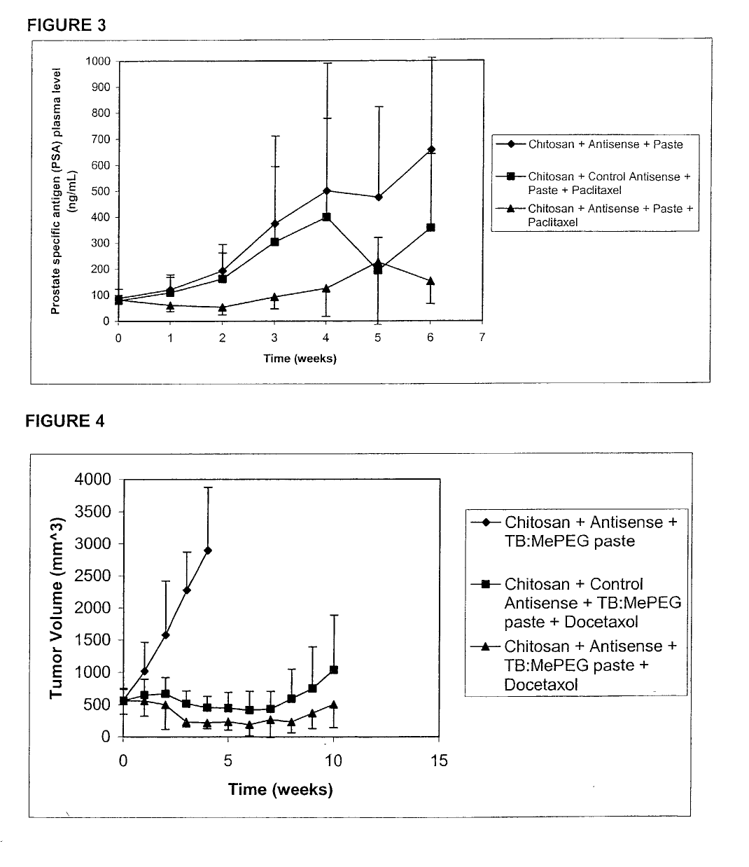

The results, as shown in FIG. 3, demonstrated that based on PSA plasma level the Chitosan+Antisense+Paste+Paclitaxel treatment resulted in tumor regression or inhibition of tumor growth for approximately 6 weeks. Each datum point represents the mean of results for a minimum of 3 mice (if more than 2-3 mice died in each group, then the data are not shown). Each error bar represents the standard deviation for its respective datum point.

Previous work using a TB:MePEG paste without chitosan or clusterin ASO but with paclitaxel required a paclitaxel loading of 10% w/w, Jackson, J. K., et al., Cancer Res. 60:4146-4151 (2000), supra, to achieve similar efficacy to the clusterin ASO-chitosan-1% paclitaxel paste in this Example, and also resulted in greater toxicities compared to this study. Thus, the present invention treated a proliferative disease using less anti-proliferative agent than without the oligonucleotide therapeutic, and decreased the side effects or toxicities.

Previous work using intraperitoneal injections of clusterin ASO and intravenous injections of paclitaxel required daily administration of the clusterin ASO for approximately two weeks followed by daily administration of the paclitaxel for approximately three weeks. Miyake, H., et al., Clin. Cancer Res. 6:1655-1663 (2000). Surprisingly, the present invention used less clusterin ASO and less paclitaxel to achieve approximately the same efficacy as the previous work. Thus, the present invention used less oligonucleotide therapeutic, anti-proliferative agent, and injections to achieve approximately the same efficacy as the protocol in the previous work. This also showed a decrease in the elimination and degradation of the oligonucleotide therapeutic.

### Example 4

The Effect of Clusterin Antisense Complexed to Chitosan Microparticles and Incorporated Into a Polymeric Paste Loaded With Docetaxol on PC-3 Human Prostate Tumors in SCID Mice.

An in vivo study was carried out using medicaments manufactured as follows: Antisense or Control Antisense was complexed to chitosan, and a Paste was prepared, as described in Example 1 and Example 2 above. Rather than incorporating Paclitaxel, Docetaxol (Taxotere®) (an anti-proliferative agent) was dissolved or suspended in appropriate pastes by physical blending in TB:MePEG paste to form a polymeric carrier:anti-proliferative agent fraction. Finally, the microparticulate:oligonucleotide therapeutic fraction was then dispersed in the polymeric carrier:anti-proliferative agent fraction by physical blending to form a homogenous paste.

Six week old SCID mice were inoculated subcutaneously in the flank region with 1×10⁶ PC-3 human prostate cancer cells and 0.1 ml Matrigel while the mice were under methoxyflurane anesthesia. When the tumors reached approximately 1 cm₃ in size the mice were randomly assigned to one of three different paste groups: 1) Chitosan complexed with Antisense (phosphorothioated clusterin ASO), incorporated into Paste (TB:MePEG paste) (that is, Chitosan+Antisense+Paste), 2) Chitosan complexed with Control Antisense (Control Antisense is a mismatch oligonucleotide, also abbreviated as MM or MM-ASO), incorporated into Paste that also contains Docetaxol (that is, Chitosan+Control Antisense+Paste+Docetaxol), and 3) Chitosan complexed with Antisense, incorporated into Paste that also contains Docetaxol (that is, Chitosan+Antisense+Paste+Docetaxol). Chitosan had a final loading in the pastes of 4% w/w, Control Antisense had a final loading in the appropriate pastes of 2% w/w, Antisense had a final loading in the appropriate pastes of 2% wlw, and Docetaxol had a final loading in the appropriate pastes of approximately 1% w/w. One hundred milligrams paste from the appropriate group was then injected into each tumor. Each group started with 6 mice. Tumor volume was measured once weekly and calculated using the formula: length×width×height×0.5236.

The results, as shown in FIG. 4, demonstrated that based on tumor volume the Chitosan+Antisense+Paste+Docetaxol treatment resulted in tumor regression or inhibition of tumor growth for approximately 10 weeks. Each datum point represents the mean of results for a minimum of 4 mice (if more than 2 mice died in each group, then the data are not shown). Each error bar represents the standard deviation for its respective datum point.

From the foregoing, it will be appreciated that, although specific embodiments have been discussed herein for purposes of illustration, various modifications may be made without deviating from the spirit and scope of the disclosure. Accordingly, the systems and methods, etc., include such modifications as well as all permutations and combinations of the subject matter set forth herein and is not limited except as by the appended claims.

What is claimed is:

1. A controlled release drug delivery composition comprising at least one polycationic polymer complexed with at least one first negatively charged pharmacologically active agent to provide controllable release of at least the first negatively charged pharmacologically active agent when administered to a patient.

2. The composition of claim 1 wherein the composition further comprises at least one pharmaceutically acceptable carrier or excipient.

3. The composition of claim 1 wherein the composition further comprises at least one pharmaceutically acceptable carrier or excipient that further comprises at least a second pharmacologically active agent.

4. The composition of claim 2 or 3 wherein the polycationic polymer comprises chitosan.

5. The composition of claim 2 or 3 wherein the first negatively charged pharmacologically active agent comprises a negatively charged oligonucleotide.

6. The composition of claim 5 wherein the negatively charged oligonucleotide comprises one or more of the group of antisense oligonucleotide, ribozyme, oligonucleotide RNA inhibitor, immune modulating oligonucleotide and nonspecific oligonucleotide.

7. The composition of claim 2 or 3 wherein the polycationic polymer comprises chitosan and the first negatively charged pharmacologically active agent comprises a negatively charged oligonucleotide.

8. The composition of claim 2 or 3 wherein the polycationic polymer comprises chitosan and the first negatively charged pharmacologically active agent comprises a negatively charged oligonucleotide and the chitosan-negatively charged oligonucleotide complex is in the form of a solution, gel, sol, suspension, spray, mousse, lotion, cream, ointment, paste, slurry, particulate, microparticulate, microsphere, film or slab within the composition.

9. The composition of claim 8 wherein the chitosan-negatively charged oligonucleotide complex is in the form of a particulate, microparticulate or microsphere within the composition.

10. The composition of claim 2 or 3 wherein the composition is a solution, gel, sol, suspension, spray, mousse, lotion, cream, ointment, paste, slurry, particulate, microparticulate, microsphere, film, slab, wrap, barrier or implant.

11. The composition of claim 10 wherein the composition is a paste.

12. The composition of claim 10 wherein the composition is a film less than about 2 mm thick.

13. The composition of claim 2 or 3 wherein the pharmaceutically acceptable carrier or excipient is a polymeric carrier.

14. The composition of claim 3 wherein the pharmaceutically acceptable carrier or excipient is a polymeric carrier that provides controllable release of at least one of the second pharmacologically active agent and the first negatively charged pharmacologically active agent.

15. The composition of claim 14 wherein the pharmaceutically acceptable carrier or excipient is a polymeric carrier that provides controllable release of the second pharmacologically active agent.

16. The composition of claim 2 or 14 wherein the composition is formulated to release greater than about 10% w/w of the first negatively charged pharmacologically active agent over a period of about 5 to 15 days.

17. The composition of claim 2 or 14 wherein the composition is formulated to release less than about 10% w/w of the first negatively charged pharmacologically active agent over a period of about 5 to 15 days.

18. The composition of claim 15 wherein the composition is formulated to release greater than about 10% w/w of the second pharmacologically active agent over a period of about 5 to 15 days.

19. The composition of claim 15 wherein the composition is formulated to release less than about 10% w/w of the second pharmacologically active agent over a period of about 5 to 15 days.

20. The composition of claim 3 or 14 wherein the second pharmacologically active agent comprises at least one of paclitaxel, docetaxol, mitoxantrone, cisplatin or methotrexate.

21. The composition of claim 20 wherein the second pharmacologically active agent comprises at least one of paclitaxel or docetaxol.

22. The composition of claim 2 or 3 wherein the composition is sized and formulated for intraperitoneal, intraarticular, intraocular, intratumoral, perivascular, subcutaneous, intracranial, intramuscular, intravenous, periophthalmic, inside the eyelid, intraoral, intranasal, intrabladder, intravaginal, intraurethral, intrarectal, adventitial, oral, nasal, rectal or topical administration to a patient.

23. The composition of claim 22 wherein the composition is sized and formulated for intraperitoneal, intraarticular, intraocular, intratumoral, perivascular, subcutaneous, intracranial, intramuscular, intravenous, periophthalmic, inside the eyelid, intraoral, intranasal, intrabladder, intravaginal, intraurethral, intrarectal or adventitial administration to a patient.

23. The composition of claim 22 wherein the composition is sized and formulated for oral, nasal or rectal administration to a patient.

24. The composition of claim 22 wherein the composition is sized and formulated for topical administration to a patient.

25. The composition of claim 2 or 3 wherein the composition is sized and formulated to be injected through a syringe needle.

26. The composition of claim 2 or 3 wherein the composition further comprises a cell permeation enhancing agent.

27. The composition of claim 2 or 3 wherein the composition further provides protection of the first negatively charged pharmacologically active agent from degradation.

28. The composition of claim 2 or 3 wherein the patient is a mammal.

29. The composition of claim 28 wherein the mammal is a human.

30. The composition of claim 29 wherein the mammal is a cow, horse, sheep, dog or cat.

31. The composition of claim 2 or 3 wherein the polycationic polymer-first negatively charged pharmacologically active agent complex is an ionic complex.

32. The composition of claim 2 or 3 wherein the polycationic polymer comprises at least one of a polyaminoacid, polyquaternary compound, protamine, polyvinylpyridine, polythiodiethylaminomethyl-ethylene, poly-p-aminostyrene, polycationic carbohydrate, polyimine, polycationic polymer derivatized with DEAE, polycationic polymethacrylate, polycationic polyacrylate, polycationic polyoxethane, polyamidoamine, polylysine, polyhistidine and polycationic starch.

33. The composition of claim 2 or 3 wherein the first negatively charged pharmacologically active agent is at least one of an anti-hepatitis agent, anti-diabetic, anti-ocular disease agent, anti-microbial, anti-viral, anti-fungal, anesthetic, anti-vascular disease agent, anti-restenotic, anti-stenotic, vasoconstrictor, vasodilator, cardiotonic, enzyme, anti-inflammatory, anti-post surgical adhesion agent, anti-psoriatic, anti-arthritic, anti-multiple sclerosis agent, anti-inflammatory bowel disease agent, hormone, bone metabolism controlling agent, hypotensive, hypertensive, sedative, anti-cancer agent, antihistamine, antitussive, vaccine, anti-neural disorder agent and asthma treatment.

33. The composition of claim 2 or 3 wherein the second pharmacologically active agent is at least one of an anti-hepatitis agent, anti-diabetic, anti-ocular disease agent, anti-microbial, anti-viral, anti-fungal, anesthetic, anti-vascular disease agent, anti-restenotic, anti-stenotic, vasoconstrictor, vasodilator, cardiotonic, enzyme, anti-inflammatory, anti-post surgical adhesion agent, anti-psoriatic, anti-arthritic, anti-multiple sclerosis agent, anti-inflammatory bowel disease agent, hormone, bone metabolism controlling agent, hypotensive, hypertensive, sedative, anti-cancer agent, antihistamine, antitussive, vaccine, anti-neural disorder agent and asthma treatment.

34. A surgical device suitable for implantation in a patient comprising a composition according to claim 2 or 3.

35. The surgical device of claim 34 wherein the surgical device is a catheter, shunt, device for continuous subarachnoid infusion, feeding tube, solid implant to prevent surgical adhesion, uterine implant, artificial sphincter, periurethral implant, splint, ophthalmic implant, contact lens, plastic surgery implant, stent including an esophageal stent, gastrointestinal stent, vascular stent, biliary stent, colonic stent, pancreatic stent, ureteric stent, urethral stent, lacrimal stent, Eustachian tube stent, fallopian tube stent, nasal stent, sinus stents, tracheal stent or bronchial stent, or a port including a venous access device comprising an external tunneled catheter, implanted port, epidural catheter or central catheter (PICC).

36. A kit comprising a composition according to claim 2 or 3 in a pharmaceutically acceptable container.

37. The kit of claim 36 wherein the kit further comprises a notice associated with the container, the notice in a form prescribed by a governing agency regulating the composition.

38. The kit of claim 36 wherein the kit further comprises instructions about at least one of use of the composition, dosing a patient or mode of administration.

39. A method of manufacturing a controlled release drug delivery composition comprising complexing at least one polycationic polymer with at least one first negatively charged pharmacologically active agent to provide controllable release of at least the first negatively charged pharmacologically active agent when administered to a patient.

40. The method of claim 39 wherein the method further comprises mixing, blending, dissolving, associating or incorporating the polycationic polymer-first negatively charged pharmacologically active agent complex with at least one pharmaceutically acceptable carrier or excipient.

41. The method of claim 39 wherein the method further comprises mixing, blending, dissolving, associating or incorporating the polycationic polymer-first negatively charged pharmacologically active agent complex with at least one pharmaceutically acceptable carrier or excipient that further comprises at least a second pharmacologically active agent.

42. The method of claim 40 or 41 wherein the polycationic polymer comprises chitosan.

43. The method of claim 40 or 41 wherein the first negatively charged pharmacologically active agent comprises a negatively charged oligonucleotide.

44. The method of claim 43 wherein the negatively charged oligonucleotide comprises one or more of the group of antisense oligonucleotide, ribozyme, oligonucleotide RNA inhibitor, immune modulating oligonucleotide and nonspecific oligonucleotide.

45. The method of claim 40 or 41 wherein the polycationic polymer comprises chitosan and the first negatively charged pharmacologically active agent comprises a negatively charged oligonucleotide.

46. The method of claim 40 or 41 wherein the polycationic polymer comprises chitosan and the first negatively charged pharmacologically active agent comprises a negatively charged oligonucleotide and the chitosan-negatively charged oligonucleotide complex is in the form of a solution, gel, sol, suspension, spray, mousse, lotion, cream, ointment, paste, slurry, particulate, microparticulate, microsphere, film or slab within the composition.

47. The method of claim 46 wherein the chitosan-negatively charged oligonucleotide complex is in the form of a particulate, microparticulate or microsphere within the composition.

48. The method of claim 40 or 41 wherein the composition is a solution, gel, sol, suspension, spray, mousse, lotion, cream, ointment, paste, slurry, particulate, microparticulate, microsphere, film, slab, wrap, barrier or implant.

49. The method of claim 48 wherein the composition is a paste.

50. The method of claim 48 wherein the composition is a film less than about 2 mm thick.

51. The method of claim 40 or 41 wherein the pharmaceutically acceptable carrier or excipient is a polymeric carrier.

52. The method of claim 41 wherein the pharmaceutically acceptable carrier or excipient is a polymeric carrier that provides controllable release of at least one of the second pharmacologically active agent and the first negatively charged pharmacologically active agent.

53. The method of claim 52 wherein the pharmaceutically acceptable carrier or excipient is a polymeric carrier that provides controllable release of the second pharmacologically active agent.

54. The method of claim 40 or 52 wherein the composition is formulated to release greater than about 10% w/w of the first negatively charged pharmacologically active agent over a period of about 5 to 15 days.

55. The method of claim 40 or 52 wherein the composition is formulated to release less than about 10% w/w of the first negatively charged pharmacologically active agent over a period of about 5 to 15 days.

56. The method of claim 53 wherein the composition is formulated to release greater than about 10% w/w of the second pharmacologically active agent over a period of about 5 to 15 days.

57. The method of claim 53 wherein the composition is formulated to release less than about 10% w/w of the second pharmacologically active agent over a period of about 5 to 15 days.

58. The method of claim 41 or 52 wherein the second pharmacologically active agent comprises at least one of paclitaxel, docetaxol, mitoxantrone, cisplatin or methotrexate.

59. The method of claim 58 wherein the second pharmacologically active agent comprises at least one of paclitaxel or docetaxol.

60. The method of claim 40 or 41 wherein the composition is sized and formulated for intraperitoneal, intraarticular, intraocular, intratumoral, perivascular, subcutaneous, intracranial, intramuscular, intravenous, periophthalmic, inside the eyelid, intraoral, intranasal, intrabladder, intravaginal, intraurethral, intrarectal, adventitial, oral, nasal, rectal or topical administration to a patient.

61. The method of claim 60 wherein the composition is sized and formulated for intraperitoneal, intraarticular, intraocular, intratumoral, perivascular, subcutaneous, intracranial, intramuscular, intravenous, periophthalmic, inside the eyelid, intraoral, intranasal, intrabladder, intravaginal, intraurethral, intrarectal or adventitial administration to a patient.

62. The method of claim 61 wherein the composition is sized and formulated for oral, nasal or rectal administration to a patient.

63. The method of claim 61 wherein the composition is sized and formulated for topical administration to a patient.

64. The method of claim 40 or 41 wherein the composition is sized and formulated to be injected through a syringe needle.

65. The method of claim 40 or 41 wherein the composition further comprises a cell permeation enhancing agent.

66. The method of claim 40 or 41 wherein the composition further provides protection of the first negatively charged pharmacologically active agent from degradation.

67. The method of claim 40 or 41 wherein the patient is a mammal.

68. The method of claim 40 or 41 wherein the mammal is a human.

69. The method of claim 68 wherein the mammal is a cow, horse, sheep, dog or cat.

70. The method of claim 40 or 41 wherein the polycationic polymer-first negatively charged pharmacologically active agent complex is an ionic complex.

71. The method of claim 40 or 41 wherein the polycationic polymer comprises at least one of a polyaminoacid, polyquaternary compound, protamine, polyvinylpyridine, polythiodiethylaminomethyl-ethylene, poly-p-aminostyrene, polycationic carbohydrate, polyimine, polycationic polymer derivatized with DEAE, polycationic polymethacrylate, polycationic polyacrylate, polycationic polyoxethane, polyamidoamine, polylysine, polyhistidine and polycationic starch.

72. A method of at least one of treating, preventing or inhibiting at least one of a proliferative disease or inflammatory disease comprising administering to a patient at least potentially having the disease a therapeutically effective amount of the composition of any one of claims 1, 2 or 3.

73. A method of at least one of treating, preventing or inhibiting at least one of a proliferative disease or inflammatory disease comprising administering to a patient, the method comprising administering a controlled release drug delivery composition produced according to any one of claims 39 to 53 to the patient.

74. The method of claim 73 wherein the composition is administered by at least one of topically, via injection through a syringe needle, intra-tumorally into a tumor, or by implanting a surgical device comprising the composition.

75. An isolated and purified composition according to any one of claims 1 to 3 for use in the manufacture of a medicament for inhibiting, preventing, or treating a proliferative or inflammatory disease in a human patient.

76. The composition of claim 75 wherein the disease is selected from the group consisting of cancer, arthritis, psoriasis or surgical adhesion.

77. The method of claim 40 or 41 wherein the method further comprises adding the composition to a surgical device suitable for implantation in a patient.

78. The method of claim 77 wherein the surgical device is a catheter, shunt, device for continuous subarachnoid infusion, feeding tube, solid implant to prevent surgical adhesion, uterine implant, artificial sphincter, periurethral implant, splint, ophthalmic implant, contact lens, plastic surgery implant, stent including an esophageal stent, gastrointestinal stent, vascular stent, biliary stent, colonic stent, pancreatic stent, ureteric stent, urethral stent, lacrimal stent, Eustachian tube stent, fallopian tube stent, nasal stent, sinus stents, tracheal stent or bronchial stent, or a port including a venous access device comprising an external tunneled catheter, implanted port, epidural catheter or central catheter (PICC).

79. The method of claim 40 or 41 wherein the method further comprises adjusting the ratio of polycationic polymer to first negatively charged pharmacologically active agent to provide a desired rate of release of the first negatively charged pharmacologically active agent from the composition.

80. The method of claim 52 wherein the method further comprises adjusting the ratio of polymeric carrier to first negatively charged pharmacologically active agent to provide a desired rate of release of the first negatively charged pharmacologically active agent from the composition.

81. The method of claim 53 wherein the method further comprises adjusting the ratio of polymeric carrier to second pharmacologically active agent to provide a desired rate of release of the second pharmacologically active agent from the composition.
